(19) United States
(12) Patent Application Publication
(10) Pub. No.: US 2007/0244184 A1
(43) Pub. Date: Oct. 18, 2007

(54) GLYCOSIDASE INHIBITORS AND METHODS OF SYNTHESIZING SAME

(75) Inventors: Brian M. Pinto, Coquitlam (CA); Nag Sharwan Kumar, Coquitlam (CA); Ramakrishna G. Bhat, Coquitlam (CA); Hui Liu, Ottawa (CA); Ravindranath Nasi, Vancouver (CA); Wang Chen, Burnaby (CA); Sankar Mohan, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: SIMON FRASER UNIVERSITY, Burnaby (CA)

(21) Appl. No.: 11/621,466

(22) Filed: Jan. 9, 2007

Related U.S. Application Data
(60) Provisional application No. 60/756,990, filed on Jan. 9, 2006.

(51) Int. Cl. A61K 31/381; A61K 31/33; A61K 31/4015; A61P 3/10; C07D 207/12; C07D 333/32; C07D 345/00

(52) U.S. Cl. 514/425; 514/183; 514/445; 540/1; 548/556; 549/66

(57) ABSTRACT

The compounds of the present invention relate to chain-extended and chain-modified analogues of salacinol, including embodiments where the sulfate moiety has been substituted with a carboxylate or phosphate moiety. In other embodiments the sulfate moiety has been shifted by one carbon atom in the zwitterionic structure. In another embodiment the polyhydroxylated side chain may be replaced with a lipophilic alkyl chain and a suitable counterion. The invention also encompasses methods for synthesizing the salacinol analogues and using the analogues for enzyme inhibition applications.

### RELATED APPLICATIONS

This application claims the benefit of U.S. Provisional Patent Application No. 60/756,990 filed 9 Jan. 2006 which is hereby incorporated by reference in its entirety.

### TECHNICAL FIELD

This application relates to zwitterionic compounds useful as glycosidase inhibitors.

### BACKGROUND

In treatment of non-insulin dependent diabetes (NIDD) management of blood glucose levels is critical. One strategy for treating NIDD is to delay digestion of ingested carbohydrates, thereby lowering post-prandial blood glucose concentration. This can be achieved by administering drugs which inhibit the activity of enzymes, such as glucosidases, which mediate the hydrolysis of complex starches to oligosaccharides in the small intestine. For example, carbohydrate analogues, such as acarbose, reversibly inhibit the function of pancreatic α-amylase and membrane-bound intestinal α-glucoside hydrolase enzymes. In patients suffering from Type II diabetes, such enzyme inhibition results in delayed glucose absolption into the blood and a smoothing or lowering of postprandial hyperglycemia, resulting in improved glycemic control.

Some naturally-occurring glucosidase inhibitors have been isolated from Salacia reticulata, a plant native to submontane forests in Sri Lanka and parts of India (known as “Kotala himbutu” in Singhalese). Salacia reticulata is a woody climbing plant which has been used in the Ayurvedic system of Indian medicine in the treatment of diabetes. Traditionally, Ayurvedic medicine advised that a person suffering from diabetes should drink water left overnight in a mug carved from Kotala himbutu wood. In an article published in 1997, Yoshikawa et al. reported the isolation of the compound salacinol from a water-soluble fraction derived from the dried roots and stems of Salacia reticulata.¹ Yoshikawa et al. determined the structure of salacinol, shown below, and demonstrated its efficacy as an α-glucosidase inhibitor. 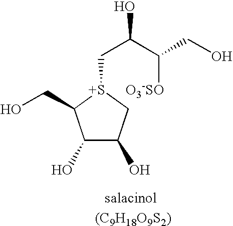
Yoshikawa et al. later reported the isolation from the roots and stems of Salacia reticulata of kotalanol which was also shown to be effective as an α-glucosidase inhibitor.² Like salicinol, kotalanol contains a thiosugar sulfonium ion and an internal sulfate providing the counterion: 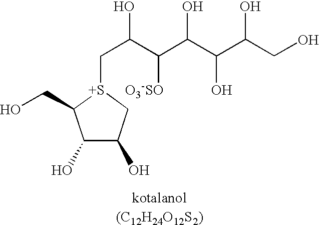
Kotalanol has been found to show more potent inhibitory activity against sucrase than salicinol and acarbose.²

The exact mechanism of action of salacinol and other glucosidase inhibitors has not yet been elucidated. Some known glycosidase inhibitors, such as the indolizidine alkaloids castanospermine and swainsonine, are known to carry a positive charge at physiological pH. 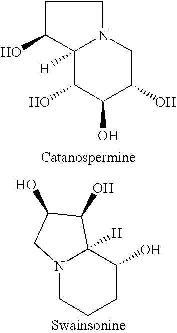
It is believed that the mechanism of action of some known inhibitors may be at least partially explained by the establishment of stabilizing electrostatic interactions between the inhibitor and the enzyme active site carboxylate residues. It is postulated that the zwitterionic compounds of the present invention, which comprise positively charged sulfonium, ammonium, and selenonium ions, could function in a similar manner. It is also possible that salacinol and other compounds of the same class may act by alteration of a transport mechanism across the intestinal wall rather than by directly binding to glucosidase enzymes.

Salacinol and kotalanol may potentially have fewer long-term side effects than other existing oral antidiabetic agents. For example, oral administration of acarbose in the treatment of Type II diabetes results in undesirable gastrointestinal side effects in some patients, most notably increased flatulence, diarrhoea and abdominal pain. As mentioned above, salacinol has been used as a therapy for diabetes in the Ayurvedic system of traditional medicine for many years with no notable side effects reported. Further, animal studies have shown that the oral ingestion of an extractive from a Salacia reticitlata trunk at a dose of 5,000 mg/kg had no serious acute toxicity or mutagenicity in rats.³

The Salacia reticulata plant is, however, in relatively small supply and is not readily available outside of Sri Lanka and India. Accordingly, it would be desirable if salicinol, kotalanol and analogues thereof could be produced synthetically from readily available starting materials rather than by extraction from natural sources. The applicant and others have previously reported the synthesis of salacinol and its stereoisomers.^{4,5,6} The applicant has also previously reported the synthesis of blintol, the selenium analogue of salacinol⁷, and ghavamiol, the nitrogen analogue of salacinol,⁸ the structures of which are shown below. 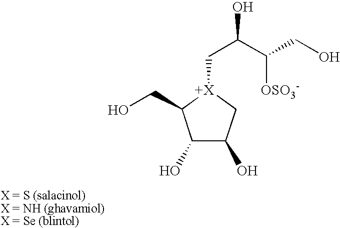
Processes for synthetically producing salacinol and its analogues are described in applicant's U.S. Pat. No. 6,455,573 issued Sept. 24, 2002 as well as pending application Ser. Nos. 10/877,490 and 11/368,014, the disclosures of which are hereby incorporated in their entireties. Both salacinol and blintol have been shown to be very effective in controlling blood glucose levels in rats after a carbohydrate meal, thus providing lead candidates for the treatment of Type II diabetes.^{7,9,56}

Structural modification of salacinol represents a promising approach in the search for new glycosidase inhibitors. The transition state structure in the enzyme-mediated hydrolysis of glycosides is believed to be an oxacarbenium ion intermediate having a distorted conformation. Strong inhibitors of glycosidase enzymes likely mimic this distorted, positively charged species. The charge distribution and stereochemical configuration of putative inhibitors appears to be important to functionality. Salacinol and some of its inhibitory analogues are zwitterions whereby the ring heteroatom is stabilized by an internal counterion. As mentioned above, in the case of salacinol, the ring sulfonium ion is stabilized by the sulfate counterion on the acyclic chain, presumably by forming a spirobicyclic-like structure.¹ The D-arabinitol configuration in the heterocyclic ring displayed by salacinol appears to be important for its activity. It is believed that interaction between the positive charge of the inhibitor and the active-site carboxylate residues on the enzyme may make a significant contribution to interaction energy.

The importance of the acyclic alditol side chain has also been investigated by the applicant and others. The fact that salacinol has greater inhibitory activity and specificity against α-glucosidases than the methyl sulfonium ion suggests that the sulfate moiety of the side chain is also functionally important.¹⁰ Yuasa et al.¹¹ have reported that docking of salacinol into the binding site of glucoamylase indicated close contacts between the sulfate ion with Arg305. Crystallographic analysis of the interactions of Drosophila melanogaster Golgi α-mannosidase II (dGMII) with salacinol and its analogues shows that the sulfate group does interact with residues in the enzyme active site.¹² The following compound, isolated from a marine sponge in Japan, has been also reported to be a strong inhibitor of α-glucosidase.¹³ The sulfate groups in this compound may play a role similar to that proposed for the sulfate group of salacinol.¹¹

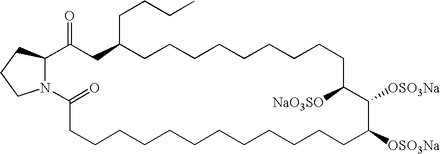

In order to develop more effective glycosidase inhibitors, there is a continuing need to synthesize and test additional salacinol derivatives, including chain-extended and chain-modified embodiments where the sulfate moiety is frame-shifted or substituted with another functional group such as carboxylate or phosphate moieties. Salicinol analogues comprising a sulfonium or ammonium ion-inner carboxylate structure represent an important new class of compounds.

The substitution of phosphate functional groups for sulfate or carboxylate groups in biologically important molecules also continues to attract much interest in bioorganic and medicinal chemistry.¹⁴ Much of the progress in this field has been associated with the phosphorus analogues of amino acids. The tetrahedral configuration, due to the presence of the phosphorus atom, allows these analogues to serve as stable analogues of the unstable tetrahedral intermediates formed in enzymatic processes. Many of these act as enzyme inhibitors. For example, N-(Phosphonoacetyl)-L-aspartate and O-Phosphate serine have been shown to be inhibitors of the enzyme carbonic anlhydrase.¹⁵ Similarly, phosphate analogues of carnitine and γ-amino-β-hydroxybutyric acid have been shown to be pharmacologically potent and purinetrione, bearing an alkyl phosphate, has been tested as an inhibitor of lumazine synthase.^{16,17}

As indicated above, another synthetic approach, instead of or in addition to modifying the acyclic chain, is to replace the ring sulphur atom with another atom, such as nitrogen. Many alkaloid sugar mimics with a nitrogen in the ring have been isolated from plants and microorganisms and inhibit various glycosidases.¹⁸⁻²⁰ For example, 1-Deoxynojirimycin, which is a D-glucose analogue with an NH group in place of the ring oxygen atom, has been shown to inhibit intestinal α-glucosidases and pancreatic α-amylase both in vitro and in vivo, as well as α-glucosidases I and II involved in N-linked oligosaccharide processing.²¹ Two N-alkylated analogues of deoxynojirimycin, namely miglitol and N-butyldeoxynojirimycin, are currently in use as drugs for the treatment of Type II diabetes and Gaucher's disease, respectively. Both drugs act by inhibition of glucosidase enzymes. 1,4-Dideoxy-1,4-imino-D-arabinitol (D-AB1), which was first isolated from the fruits of the legume Angylocalyx boutiquenus, was found to be a potent inhibitor of hepatic glycogen phosphorylase²² and its synthetic L-enantiomer (L-AB1) is a powerful inhibitor of mammalian α-D-glucosidases.^{23,24} The naturally occurring glycosidase inhibitor acarbose,²⁵ which contains a nitrogen atom in one of the linkages between the sugar and pseudosugar units, is the highest-affinity carbohydrate analogue for a binding protein, and has also been used for the treatment of Type II diabetes as indicated above.^{26,27} It is generally believed that this strong binding originates from electrostatic interactions of the positively charged, protonated nitrogen atom with carboxylate residues in the enzyme active-site.²¹ A similar mode of action has been suggested for the naturally occurring indolizidine alkaloids castanospermine and swainsonine, as mentioned above.

N-alkylated imino sugars have also been extensively studied in recent years due to their potential use as glycosidase inhibitors. Generaly speaking, N-alkylation increases the glycosidase inhibitory activity of the parent imino sugar.²⁸⁻³⁰ For example, N-methyl-, N-butyl-, N-decyl- and N-(7-oxadecyl)-1-deoxynojirimycin are more potent inhibitors of porcine liver α-glucosidase I than the parent compound, 1-deoxynojirimycin (DNJ).³¹ Although the exact role of the alkyl chain in increasing inhibition is not well understood, biochemical characterization of N-alkylated imino sugars indicated that the lipophilic alkyl chains play a role in the cellular uptake of the inhibitor.³² In a recent study of the molecular requirements of these compounds for glycosidase inhibition, it was reported that the protonated imino sugar mimics the charge on the proposed oxacarbenium-ion transition state formed during hydrolysis of the natural substrate.³³ In addition, it has been proposed that the deprotonation of the imino sugars in the slightly basic pH (7.1) of the ER, resulting in a loss of cationic properties, could be one of the possible reasons for the much lower in vivo glycosidase activity exhibited by the N-butyl compound than in the in vitro studies.³³ Hence, it is of interest to design inhibitors that incorporate a permanent positive charge at a suitable position as possible substitutes for the N-alkylated imino sugars.

In the same maimer, sulfonium-ion compounds may be modified in order to increase the required electrostatic interactions between the inhibitor and an active-site carboxylate residue. In addition to glycosidase inhibitors, S-alkylated sulfonium-ions could act as glucosyltransferase inhibitors by analogy with their N-alkylated imino sugar counterparts. For example, N-butyl-1-deoxynojirimycin was found to be not only an α-glucosidase I inhibitor but also a potent inhibitor of ceramide-specific glucosyltransferase, a key enzyme involved in the biosynthesis of glycosphingolipids.³³ Increases in alkyl chain lengths have led to increases in transferase inhibitory activities of these N-alkylated imino sugars, suggesting a hydrophobic environment is part of substrate recognition. Since, the glycosyl transfer is also believed to proceed through a transition-state with substantial oxacarbenium-ion character, by simple analogy with the well-studied mechanism of glycosidases,³⁴ sulfonium-ion compounds bearing a permanent positive charge on the sulfur atom could provide the necessary electrostatic interactions in the enzyme active site together with the attached lipophilic alkyl chains required for substrate recognition.

Since glycosidases are involved in many different biological processes, such as digestion, the biosynthesis of glycoproteins, and the catabolism of glycoconjugates, the compounds of the present invention may find application in a wide variety of biological applications. Various glycosidase inhibitors have shown antiviral, insect antifeedant, antidiabetic and anticancer effects as well as immune modulatory properties. With respect to anticancer effects, tumor cells often display very complex oligosaccharide structures that are usually found in embryonic tissues. It is believed that these complex structures provide signal stimuli for rapid proliferation and metastasis of tumor cells. A possible strategy for therapeutic use of glucosidase inhibitors is to take advantage of the differential rates of normal vs cancer cell growth to inhibit assembly of complex oligosaccharide structures. For example, the indolizidine alkaloid swainsonine, an inhibitor of Golgi α-mannosidase II, reportedly reduces tumor cell metastasis, enhances cellular immune responses, and reduces tumor cell growth in mice.³⁵ Swainsonine treatment has led to significant reduction of tumor mass in human patients with advanced malignancies, and is a promising drug therapy for patients suffering from breast, liver, lung and other malignancies.^{36,37}

The compounds of the present invention may also find application in the treatment of Alzheimer's disease due to their stable, internal salt structure. Alzheimer's is characterized by plaque formation in the brain caused by aggregation of a peptide, β-amyloid, into fibrils. This is toxic to neuronal cells. One can inhibit this aggregation by using detergent-like molecules. It is believed that the compounds of the present invention, which are amphipathic, may demonstrate this activity.

There is a continuing need for new glycosidase inhibitors which may be synthesized in high yields from readily available starting materials and which have potential use for therapeutic and other applications.

### SUMMARY OF THE INVENTION

The compounds of the present invention relate to chain-extended and chain-modified analogues of salacinol, including embodiments where the sulfate moiety has been substituted with a carboxylate or phosphate moiety. In other embodiments the sulfate moiety has been shifted by one carbon atom in the zwitterionic structure. In another embodiment the polyhydroxylated side chain may be replaced with a lipophilic alkyl chain and a counterion. The invention also encompasses methods for synthesizing the salacinol analogues and using the analogues for enzyme inhibition applications.

In one embodiment the invention relates to a non-naturally occurring compound selected from the group consisting of compounds represented by the general formula (I) and pharmaceutically acceptable salts thereof: 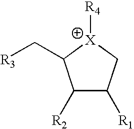
where X is selected from the group consisting of S, Se and NH; R₁, R₂, and R₃ are the same or different and are selected from the group consisting of H, OH, SH, NH₂ and halogens and R₄ is selected from the group consisting of a polyhydroxylated acyclic alkyl chain comprising a anionic sulfate, carboxylate or phosphate moiety and a lipophilic alkyl chain between 2 and 20 carbons in length with an external counterion.

### BRIEF DESCRIPTION OF THE DRAWINGS

In drawings which are intended to illustrate embodiments of the invention and which are not intended to limit the scope of the invention:

FIG. 1 is a stereoview of compound 21 in the active site of of Drosophila melanogaster Golgi Mannosidase II. and its surrounding electron density. The electron density was determined as a simulated annealing omit map (Fo-Fc) and is contoured at 2 sigma (left view) or 5 sigma (right view). The active site zinc ion is denoted by a grey circle.

FIG. 2 shows interactions of compound 21 with Drosophila GMII. Only interactions less than 3.2 Å are indicated. The zinc ion in the active site is depicted as a black ball and water molecules are shown as grey spheres. Distances are given in Angstrom units. Numbering of the inhibitor is at it occurs in the PDB file.

FIG. 3 is an overlay of compounds bound in the active site of dGMII. Compound 21 is overlayed with A. Swainsonine (PDB 1HWW) B. Ghavamiol 2 (PDB 1TQU) C. Salacinol Diastereomer (PDB 1TQT) or D. N-Benzyl mannostatin (PDB 2F7P).

FIG. 4 depicts NOE correlations observed in the 1D-NOESY spectra of compounds 71 and 73.

FIG. 5 shows IC₅₀ values for various N-alkylated imino sugars against porcine liver α-glucosidase I.

### DETAILED DESCRIPTION OF THE INVENTION

Salacinol is a naturally occurring compound which may be extracted from the roots and stems of Salacia reticulata, a plant native to Sri Lanka and India. Synthetic routes for preparing Salacinol (1), and its nitrogen (2) and selenium (3) analogues shown below have been previously described in U.S. Pat. No. 6,455,573 issued Sep. 24, 2002 as well as pending U.S. application Ser. Nos. 10/877490 and 11/368,014, the disclosures of which are hereby incorporated in their entireties. As indicated above, the nitrogen and selenium analogues of salacinol have been ascribed the names ghavamiol and blintol respectively. 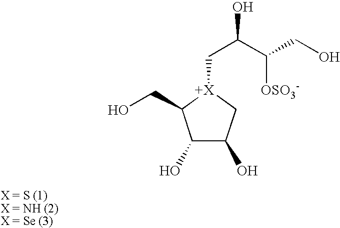
This application relates to synthetic routes for preparing derivatives of compounds (1) to (3), including chain-modified and chain-extended analogues.
1.0 Carboxylate Analogues
1.1 Sulfonium Carboxylate Analogues

As discussed above, the acyclic side chain of salicinol and kotalanol includes a sulfate group which is believed to be important to the inhibitory activity of these compounds. In embodiments of the present invention the sulfate moiety has been substituted with a carboxylate moiety. In a first embodiment, target compounds 4 and 5 comprise a thioarabinitol having a polyhydroxylated side chain containing a carboxylate residue.

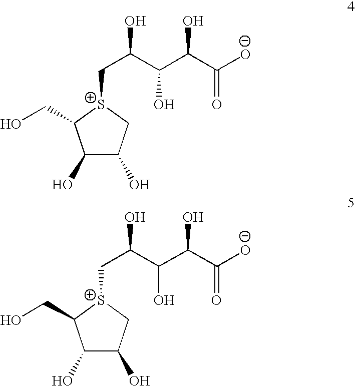

With reference to Scheme 1, retrosynthetic analysis indicated that carboxylate analogues of salacinol could be obtained by alkylation of thioarabinitols at the sulfur atom. The alkylating agent could be an epoxide, whereby regioselective attack of the sulfur at the least hindered primary center should afford the desired sulfonium ions.³⁸ As discussed below, the epoxide may be synthesized from a ribonolactone.

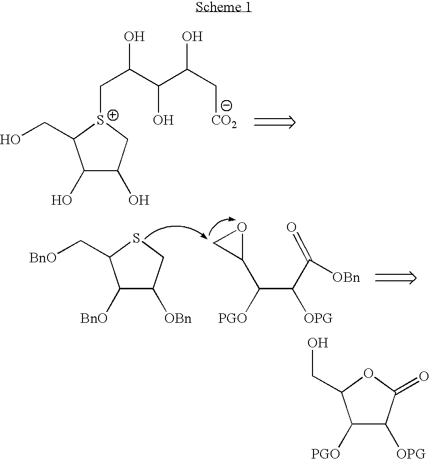

Scheme 2 below shows an exemplary means for synthesizing the thioarabinitol reagents. Thioarabinitol 8 and 11 were synthesized from D-xylose and L-xylose respectively, following a similar strategy previously published by the inventors.⁶ Treatment of the diol 6 with methanesulfonyl chloride in pyridine afforded the dimesylate 7 in 88% yield. Treatment of 7 with sodium sulfide in DMF produced compound 8 in 95% yield. Compound 11 may be produced in a similar manner from diol 9 and dimesylate 10 (Scheme 2).

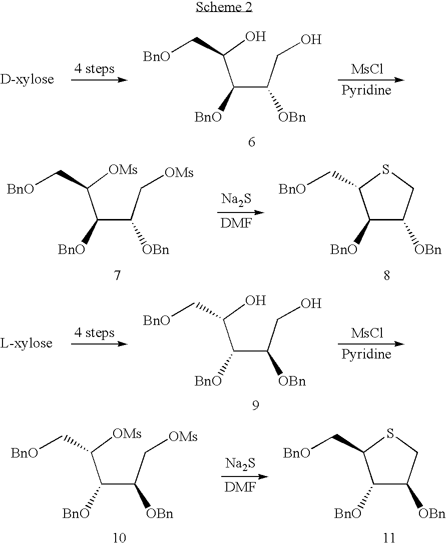

The expoxide reagent may be synthesized as shown in Scheme 3. D-Ribonolactone 12 was converted to the 2,3-O-isopropylidene-D-ribonolactone 13,³⁹ that was tosylated to give compound 14 in 88% yield (Scheme 3). Treatment of 14 with sodium benzylate afforded the desired epoxide 15 in 86% yield.⁴⁰

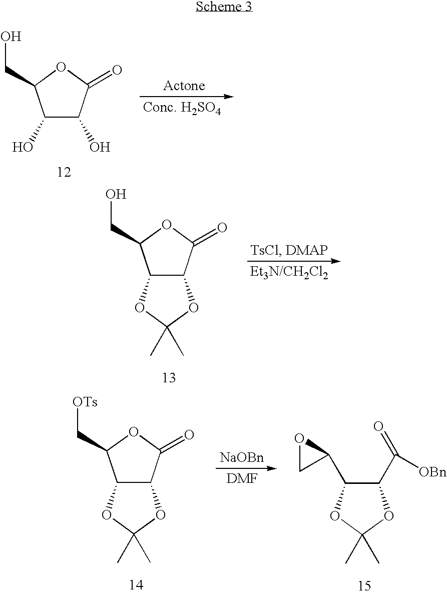

Schemes 4 and 5 below illustrate the coupling reactions for reacting the thioarabinitol and epoxide reagents. As shown in Scheme 4, regioselective ring opening of the epoxide 15 by the nucleophilic attack of the sulfur atom in the thioether 8 occurred rapidly in a mixture of CF₃COOH and CH₂Cl₂ to give 16 in 58% yield. Even after purification of compound 16 by flash chromatography, ¹H NMR and ¹³C NMR spectra showed some extra peaks. These peaks must be caused either by an inseparable impurity or by the presence of a similar compound with another external negative counterion. The compound was therefore processed with an additional purification as follows for the purpose of characterization. Counterion exchange and deprotection of the hydroxyl groups on the side chain were achieved in a mixture of concentrated hydrochloric acid and methanol. The resulting compound 17 was purified by flash chromatography and characterized by ¹H NMR, ¹³C NMR, COSY, and HMQC. Hydrogenolysis of the coupled compound 16 over Pd/C catalyst did not go as planned to give compound 4 (Scheme 4) because of poisoning of the catalyst. Debenzylation of the protected compound 16 was therefore accomplished by treatment with boron trichloride in CH₂Cl₂, affording 4 in 50% yield.

As shown in Scheme 5, compound 5, the diastereomer of 4, was similarly obtained by reaction of the thioether 11 with the epoxide 15 to produce the protected compound 18 in 66% yield. Counterion exchange and deprotection of the hydroxyl groups on the side chain gave compound 19 in 75% yield. Compound 19 was fully characterized as indicated in the experimental section below. Stereochemistry at the sulfur atom was determined by a 1D-NOE experiment. Treatment of 18 with boron trichloride in CH₂Cl₂ afforded compound 5 in 52% yield.

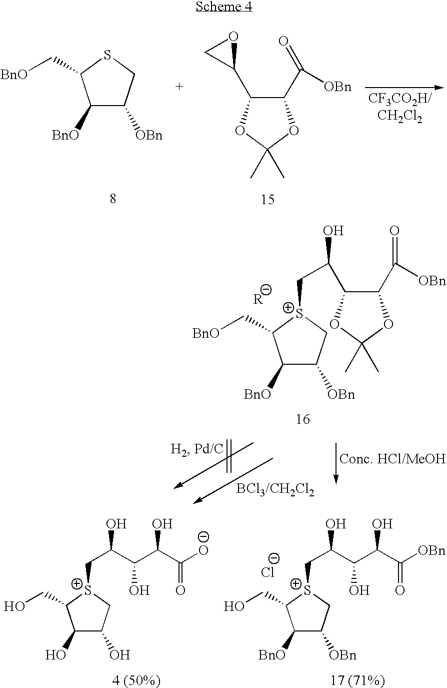

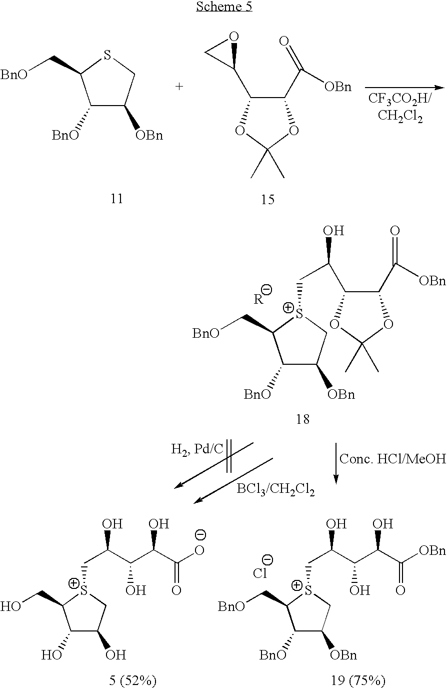

The inhibitory activity of target compounds 4 and 5 was assessed in respect of recombinant human maltase glucoamylase (MGA). Only compound 5, with the D-arabinitol configuration in the heterocyclic ring displayed by salacinol 1, was found to be active, with a Ki value of 10 μM.⁴¹

1.2 Nitrogen Carboxylate Analogues

In a second embodiment of the invention, nitrogen analogues of salacinol having a polyhydroxylated side chain containing a carboxylate residue were synthesized. Target compounds 20 and 21 are chain-extended and chain-modified versions of ghavamiol 2.

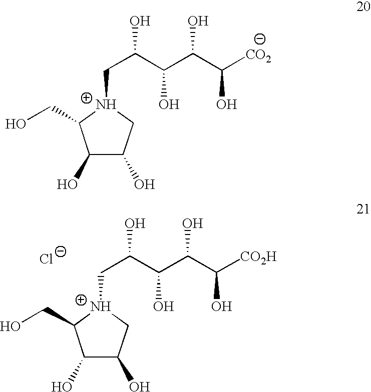

With reference to Scheme 6, retrosynthetic analysis indicated that carboxylate analogues of salacinol could be obtained by alkylation of iminoarabinitols at the nitrogen atom in a manner similar to Scheme 1. The alkylating agent could be an epoxide whereby regioselective attack of the amine at the least hindered primary center should afford the desired amino acids.⁴² In this example, the epoxide could be synthesized from inexpensive Vitamin C as described below.

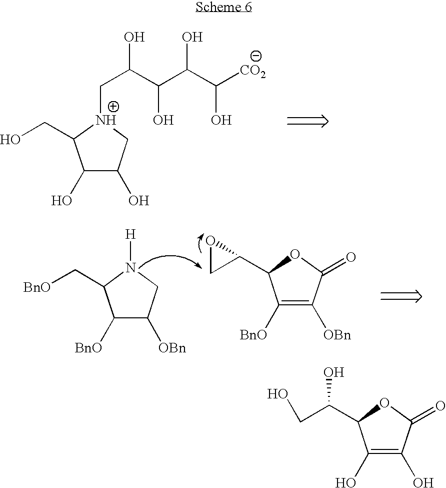

In this embodiment the epoxide reagent may be synthesized as shown in Scheme 7 using a simplified procedure of Raic-Malic et al.⁴³ The iminoarabinitols 28 and 31 were synthesized from D-xylose and L-xylose, respectively, following a strategy previously published by the inventors (Scheme 8).^{6,44,45}

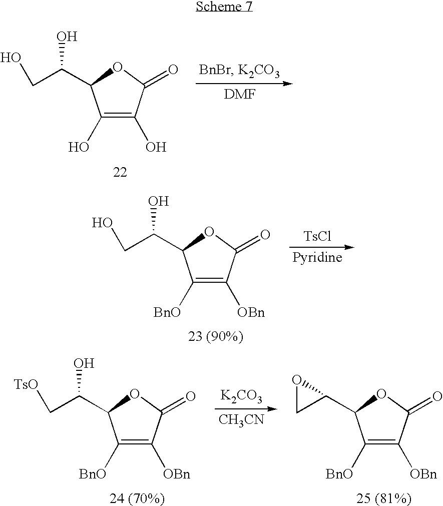

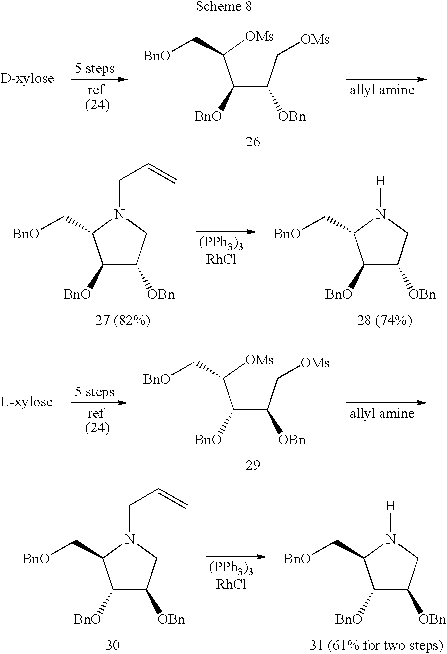

Coupling of 2,3,5-tri-O-benzyl-1,4-dideoxy-1,4-imino-L-arabinitol 28 with the benzyl-protected L-ascorbic acid epoxide 25 in dry acetonitrile at 70° C. gave the protected compound 32 in 82% yield (Scheme 9). No side products were obtained. Debenzylation of the coupled product 32 by hydrogenolysis and subsequent stereoselective catalytic reduction of the C4′-C5′ double bond of the L-ascorbic acid moiety, using a widely employed procedure,⁴⁶⁻⁴⁸ afforded 33. Catalytic hydrogenation of the L-ascorbic acid was reported to proceed with complete diastereoselectivity.^{46,47} The reduction of the double bond in 32 was monitored by MALDI-TOF mass spectrometry as a hydrogen chloride salt. Even though a high pressure of H₂ was used, the reduction was complete only after 4 days. Without further purification, the crude compound 33 was treated with aqueous potassium carbonate. After hydrolysis of the lactone ring in 33 and neutralization of potassium carbonate with acid, the resulting inorganic salts were removed using Sephadex G-10 chromatography to yield target zwitterions compound 20. The overall yield for the two steps was 78%. The structure of the zwitterion 20 was confirmed by MALDI-TOF mass spectrometry, microanalysis data, and ¹H and ¹³C NMR spectroscopy.

Compound 21, the diastereomer of 20, was similarly obtained by reaction of the amine 31 with the epoxide 25 to produce the protected compound 34 in 74% yield (Scheme 10). Deprotection, stereoselective catalytic reduction, and hydrolysis, and exchange of Na⁺ ion with excess cation exchange resin gave compound 21 in 62% yield. In this case, the compound was obtained as a chloride salt, as confirmed by MALDI-TOF mass spectrometry, microanalysis data, and ¹H and ¹³C NMR spectroscopy.

As indicated in the experimental section set out below, NMR spectra were performed on samples of compounds 20 and 21 in deuterated water, made basic with small amounts of sodium deuteroxide to give the corresponding amines, to ensure the peaks were more defined. The inventors believe that the broadening of the peaks in the absence of base is due to chemical exchange between the ammonium salts and the corresponding tertiary airlines, a process that is in the intermediate-exchange regime on the NMR time scale. In the presence of base, only the rapidly inverting tertiary amines are present, and conformationally averaged NMR spectra in the fast-exchange regime are observed.

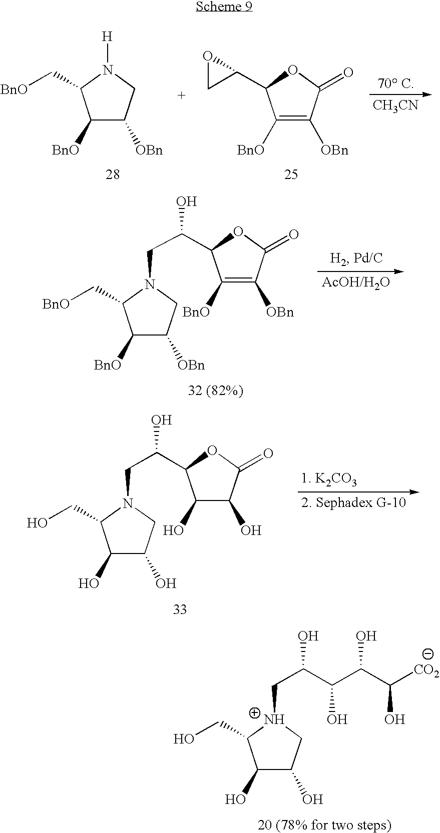

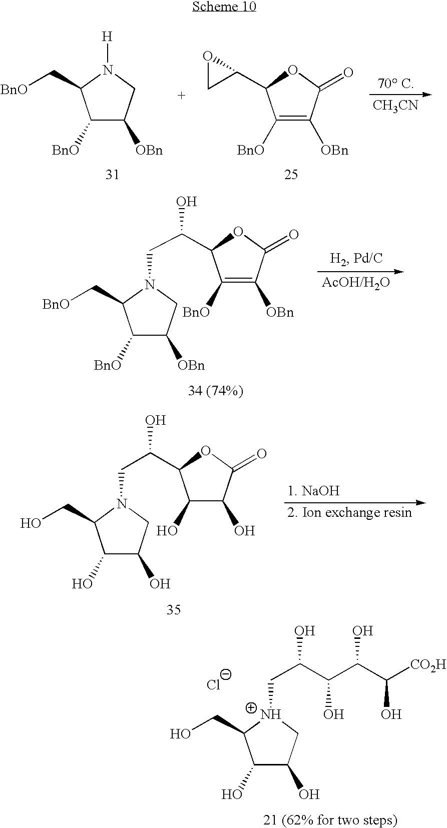

The inventors measured the enzyme inhibitory activity of the amino acids 20 and 21. Compound 21 inhibits recombinant human MGA with a Ki value of 21 μM. Salacinol 1 itself has a Ki value of 0.2 μM. Compound 21 is also active against Drosophila melanogaster Golgi α-mannosidase II (dGMII) with an IC₅₀ of 0.3 mM. This is a significant improvement (25-fold) over the inhibition measured for salacinol and kotalanol as well as other salacinol analogues such as blintol which all inhibited dGMII with an IC₅₀ of approximately 7.5 mM.¹² Compound 20 is not active on either enzyme; similar results were obtained for other salacinol analogues derived from anhydro-L-heteroarabinitol moieties and presumably reflect minimal contacts of the enantiomeric five-membered rings in the enzyme active sites.^{8,12}

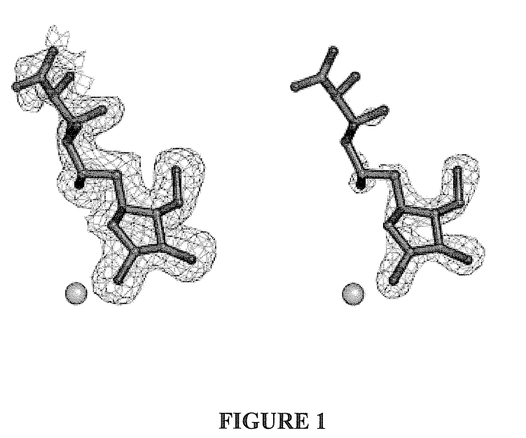

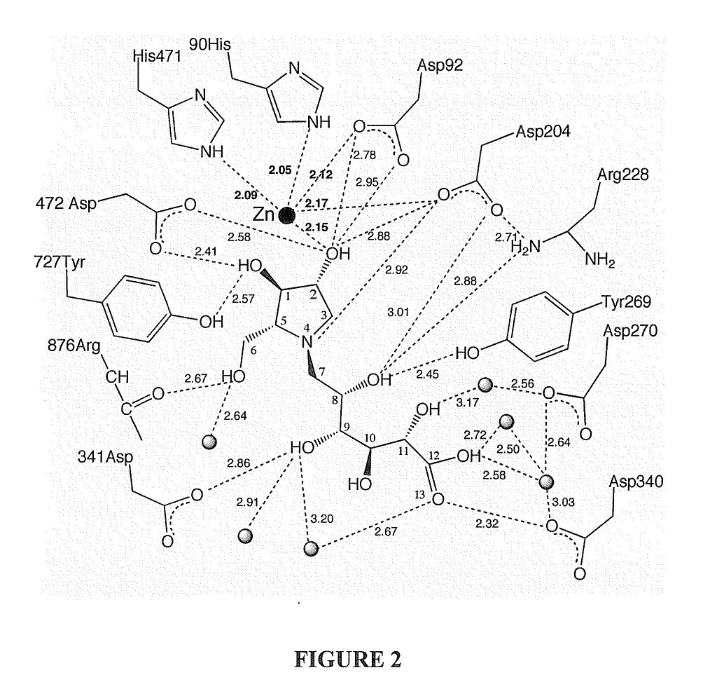

The crystal structure of compound 21 bound in the active site of dGMII was also determined. Statistics for data collection and refinement are presented in Table 1. The electron density of the bound compound 21 is shown in FIG. 1. Close interactions (with a distance of less than 3.2 Å) are highlighted in FIG. 2. Similar to the other salacinol analogues (and in contrast to most other inihibitors bound to dGMII) only a single hydroxyl group (OH2) interacts with the active site zinc atom. Also, as seen in other salacinol analogues, Tryptophan95 stacks on top of the ring portion of 21, and the ring hydroxyl groups form hydrogen bonds with aspartate residues (D92, D204, and D472 with OH1 and D472 with OH2) and tyrosine (Y727 OH with OH2). The C6 OH forms hydrogen bonds with the carbonyl oxygen of R876 as well as a bound water molecule. D204 makes a hydrogen bond with the nitrogen atom of compound 21.

It is in the acyclic chain of 21 that the interactions of the inhibitor exhibit the most significant differences from the other salacinol analogues, and it is these novel interactions which may account for the increased potency of 21 in comparison to its parent compound ghavamiol 2. The hydroxyl groups of this chain form extensive contacts with both side-chains and water molecules in the active site. The electron density in this region is more ill-defined than that of the ring region and this indicates that there is mobility of the chain. Flexibility of this region is also reflected in the temperature (B) factors which are a measure of atomic mobility. B-factors in the ring region are in the range of 10-15 Å² (the zinc bound OH1 is below 10 Å²) while those in the tail region approach 44 Å².

Hydrogen bonds in the acyclic region occur between O8 and the catalytic nucleophile D204, as well as R228 and Y269. O9 interacts with the acid-base catalyst residue D341 and two waters. O11 hydrogen bonds to one water while O12 makes close contacts with two waters. The carbonyl O13 interacts with D340 and 2 water molecules, one of which is shared with O9 and the other shared with O12, D340 and D270.

TABLE 1
Statistics for Data Collection and Refinement

| PDB code/HET symbol | 2FYV/W72 |
|---|---|
| Space Group | P2_{I}2_{I}2_{I} |
| Cell dimensions (Å) | 68.81 × 108.64 × 137.38 |
| Data Collection (values in parenthesis | |
| represent high resolution shell) | |
| Resolution (Å) | 30-1.90 (1.95-1.90) |
| Unique Reflections/Redundancy | 81152/5.7 (5388/2.7) |
| I/sigma I | 12.4 (2.5) |
| % Completeness | 99.6 (95.4) |
| R merge | 0.092 (0.41) |
| Wilson B (Å²) | 13.3 |
| Structure Refinement | |
| R_{workt}/R_{free} (reflections for R_{free}) | 0.162/0.212 (1848) |
| Amino Acids/Alternate Conformers | 1044/10 |
| Water Molecules/HeteroAtoms | 1048/49 |
| rmsd bonds (Å)/rmsd angles (°) | 0.2/1.9 |
| Average B Factors (Å²) | |
| Overall | 16.8 |
| Protein Main Chain/Side Chain | 14.5/16.4 |
| Water | 26.7 |
| Inhibitor(range) | 25.6 (10-44) |
| Zn/MPD/PO₄ | 10.8/21.4/42.9 |

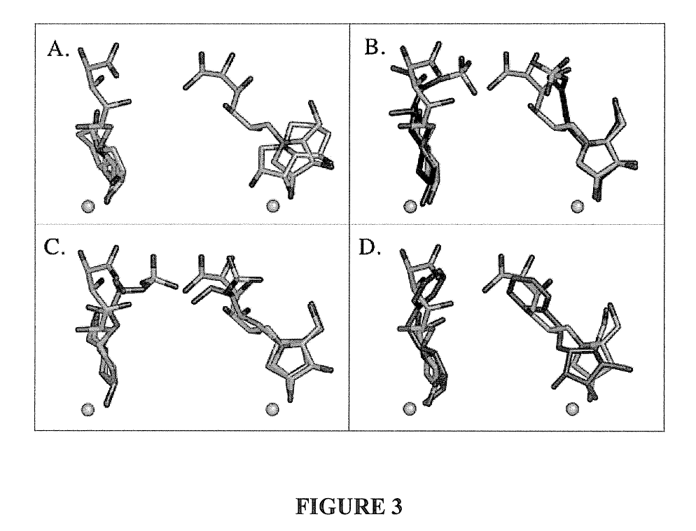

FIG. 3A shows an overlay of compound 21 and swainsonine (from PDB 1HWW) bound in the crystal structure of dGMII . Although the reason for the potency of swainsonine has not been clearly determined, it is one of the best inhibitors of dGMII, with an IC₅₀ value in the range of 20-40 nM, and is believed to closely mimic the oxacarbenium ion which occurs in the reaction pathway. The position of the nitrogen moiety, which is designed to serve as the mimic of the positive charge on the oxacarbenium ion, is almost identical in the two bound structures. However, in contrast with swainsonine, only a single hydroxyl group of 21 is in contact with the active site zinc ion. As well, the orientation of the second hydroxyl group, which forms hydrogen bonds with D472 and Y727, differs in the two structures, and it is possible that this geometry is not ideal for forming strong interactions. While the position of the head group of 21 is comparable to the other salacinol analogues¹² the region of space occupied by the acyclic tail region is quite different. FIG. 3 shows overlays of 21 bound in the crystal structure of dGMII with bound ghavamiol 2 (FIG. 3B) or the diastereomer of salacinol 32 (FIG. 3C) solved previously (PDB's 1TQU and 1TQT¹²). In both cases the position of the sulfate group is quite different from the carboxyl group, and the space through which the aliphatic chain passes is also quite different. Interestingly, the region of the active site through which the aliphatic chain of 21 passes is very comparable to that of a recently solved benzyl-Mannostatin A:dGMII complex.⁴⁹ The overlay of the two complexes (FIG. 3D) shows them to intertwine, although the nature of the interactions formed by the two tail moieties is different. The benzyl tail reduced the potency of the mannostatin to which it was attached⁵⁰ while in the present case the carboxylate tail greatly increased the inhibitory activity of the salacinol head group.

2.0 Phosphate Analogue

In a further embodiment of the invention the sulfate moiety on the acyclic side chain has been substituted with a phosphate moiety. In this embodiment, phosphorylated heteroalditols 36 and 37 were synthesized. Compounds 36, 37 are new analogues of natural inhibitors such as N-hydroxyalkyl derivatives of polyhydroxy pyrrolidines and salacinol 1.

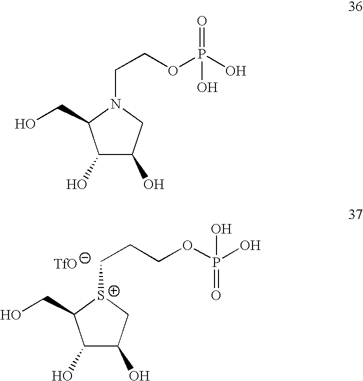

The desired target compounds (36, 37) were synthesized by pursuing the Mitsunobu reaction of N- and S-hydroxyalkyl derivatives of heteroalditols with dibenzyl phosphate, followed by catalytic hydrogenolysis. With reference to Scheme 11 below, the required dimiesylate (38) was prepared from commercially available L-xylose in five steps (68%) following a reported procedure.⁵¹ Further, N-hydroxyethyl-2,3,5-tri-O-benzyl-1,4-dideoxy-1,4-imino-D-arabinitol (39) was synthesized in 83% yield from the reaction of the dimesylate (38) with ethanolamine following a literature procedure⁶ for the synthesis of N-allyl-iminio-D-arabinitol (Scheme 11).

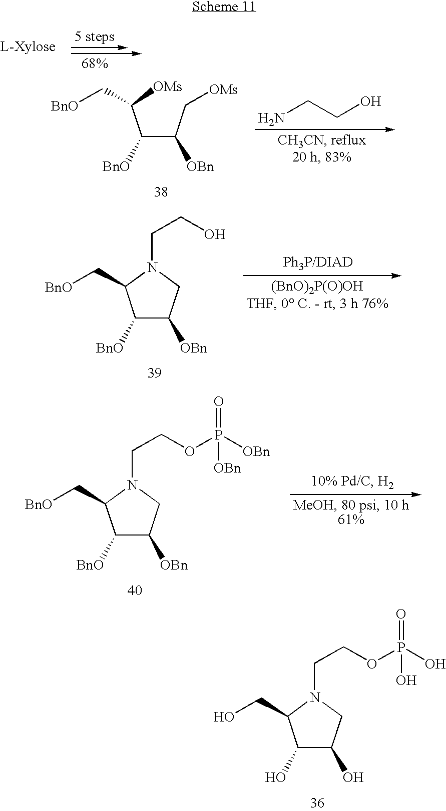

Treatment of the compound (39) with dibenzyl phosphate under Mitsunobu reaction conditions furnished the corresponding 2′-phosphorylated derivative of N-hydroxyethyl-1,4-Dideoxy-1,4-imino-D-arabinitol (40) in 76% yield (Scheme 11). Finally, hydrogenolysis of (40) using 10% Pd on carbon afforded the desired product (36) as a white solid in 61% yield. Compound (36) was characterized by ¹H, ¹³C, gCOSY, gHMQC NMR, MALDI-TOF and elemental analysis.

Similarly, for the synthesis of (37), the required compound, 2,3,5-tri-O-benzyl-1,4-anhydro-1,4-thio-D-arabinitol (41) was prepared from the commercially available L-xylose in six steps, in an overall yield of 67% (Scheme 12).⁵¹

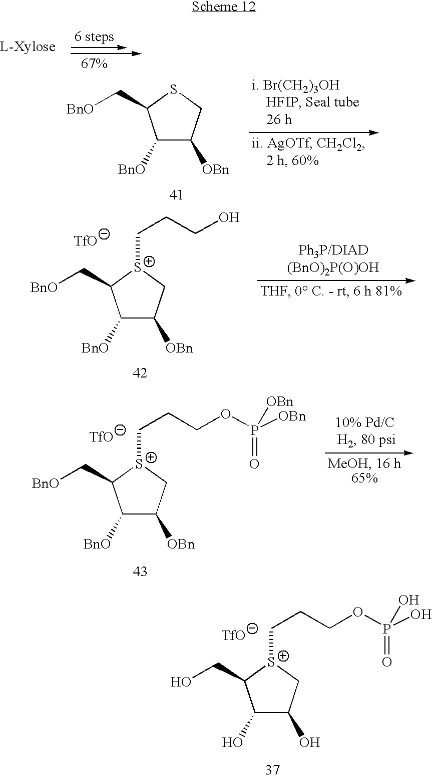

The alkylation reaction was then examined. Thus, reaction of 41 with 3-bromo-1-propanol in 1,1,1,3,3,3-hexafluoro isopropanol (HFIP), followed by treatment of the reaction mixture with silver triflate afforded the hydroxypropyl-2,3,5-tri-O-benzyl-1,4-anhydro-1,4-thio-D-arabinitol (42) in 60% yield.

Preparation of the phosphate (43) was important from a synthetic point of view as the mode of addition of reactants was found to be a determining factor for the efficient synthesis of compound (43). Initially, a mixture of triphenylphosphine, compound (42) and dibenzyl phosphate in anhydrous THF at 0° C. for 5-10 min was stirred, and diisopropyl azodicarboxylate (DIAD) was added dropwise, in accordance with the literature procedure.⁵² However, the desired product was not obtained. The inventors suspected that triphenylphosphine must have reacted with the sulfonium salt. A second set of conditions that involved initial stirring of triphenylphosphine and DIAD in dry THF for 10 min followed by the gradual addition of dibenzyl phosphate, then compound (43), resulted in the consumption of 50% of the substrate. Finally, stirring triphenylphosphine and DIAD in dry THF at 0° C. for a short time (2-3 min), followed by the immediate addition of dibenzyl phosphate, followed, in turn, by addition of a solution of (42) in anhydrous THF dropwise at 0° C., and stirring for 6 h at room temperature furnished the desired product (43) in 81% yield. Debenzylation was carried out by high-pressure hydrogenolysis using 10% Pd on carbon to obtain the thio-alditol phosphate (37) as a viscous oil in 65% yield. Compound (37) was characterized by ¹H, ¹³C, gCOSY, gHMQC NMR, MALDI-TOF and elemental analysis. The stereochemistry at the sulfur centre was assigned with the aid of a NOESY experiment which showed a correlation between H-4 and H-1′, suggesting that the side chain and the C-4 substituent were trans to one other.

After numerous attempts under variety of reaction conditions the inventors failed to synthesize compound (43) from the thioarabinitol (41) and 3-bromopropyl-dibenzyl phosphate, as the nucleophilic sulfur atom attacked the benzyl group on the phosphate to furnish the salt, S-benzyl-2,3,5-tri-O-benzyl-1,4-dideoxy-1,4-thio-D-arabinitol bromide.

3.0 S-Alkylated Analogues

In a further embodiment of the invention, the inventors have synthesized salacinol derivatives bearing a permanent positive charge of the sulphur atom and including a lipophilic alkyl chain which may be important for substrate recognition. The target compounds 44-52 include the 1,4-anhydro-4-thio-D-arabinitol moiety.

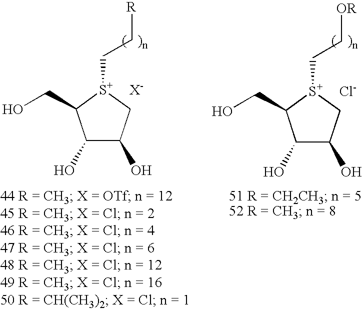

As shown generally in Scheme 13, the sulfonium-ions could be synthesized by alkylation of an appropriately protected anhydrothio-D-arabinitol at the ring sulfur atom using alkylating agents (53-58, 60 and 62).

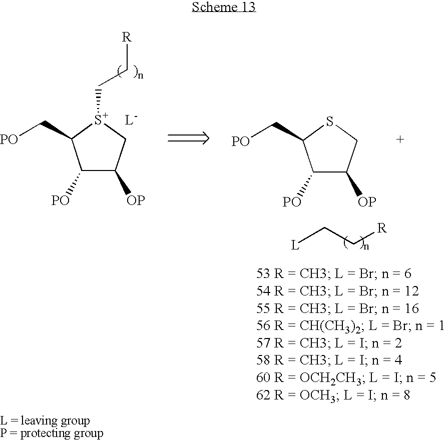

The required alkyl bromides 53-56 and alkyl iodides 57 and 58 were commercially available. The alkyl iodides 60 and 62 were synthesized in three steps starting from commercially available 6-bromohexan-1-ol (59) and 9-bromononan-1-ol (61), respectively, as shown in Scheme 14. The bromo alcohol 59 was treated with NaOEt in refluxing EtOH for 5 h to produce the ethoxy alcohol that was subsequently converted into the corresponding mesylate. The crude mesylate was then treated with NaI in dry acetone to produce the corresponding iodide 60 that was purified by chromatography and used immediately in the alkylation reaction. Similarly, the iodide 62 was prepared from the bromo alcohol 61, except that NaOMe in refluxing MeOH was used in the first step (Scheme 14). The required 1,4-anhydro-2,3 5-tri-O-benzyl-4-thio-D-arabinitol (63) was prepared from L-xylose, as described by Satoh et al.⁵¹ for the synthesis of its enantiomer.

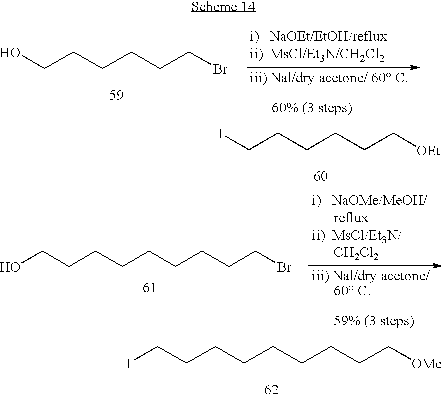

Initially, the S-alkylation of compound 63 with n-tetradecyl bromide 54 was examined in order to optimize the reaction conditions for the alkylation reaction. At first, the reaction was carried out at room temperature in 1,1,1,3,3,3-hexafluoroisopropanol (HFIP), with 63 and 54 present in a 1:1 ratio. After 24 h of continuous stirring in a sealed tube at room temperature, there was no product formation observed, as indicated by TLC. Increasing the reaction temperature to 90° C. and stirring the reaction mixture for 24 h in a sealed tube resulted in ³⁰% of the desired product 64 that was purified by column chromatography. As the S-alkylated sulfonium ion 64 formed in this reaction was deemed to be unstable due to the ring opening reaction by the bromide counterion, after column chromatographic purification, the product 64 was treated immediately with AgOTf (1 equiv) in CH₂Cl₂ to exchange the bromide counterion with triflate, resulting in the stable S-alkylated sulfonium ion 65 (Scheme 15).

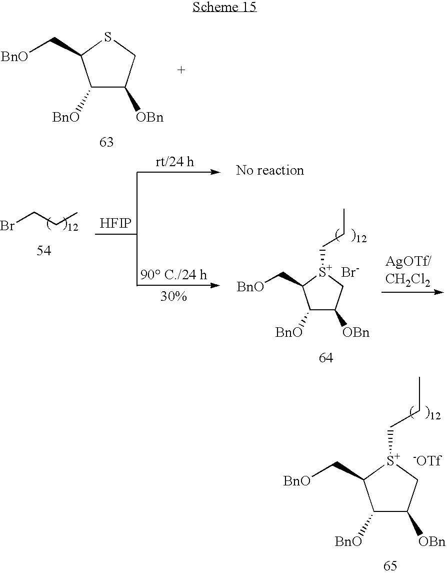

Attempts were made to improve the yield of this reaction by the addition of AgOTf (1 equiv. with respect to alkyl bromide) initially to the reaction mixture in HFIP. Surprisingly, there was no product formation, and decomposition of the starting material 63 was observed after stirring at 90° C. for 24 h, as indicated by TLC. Based on a literature precedent,⁵³ the reaction was repeated with AgBF₄ (1 equiv. with respect to alkyl bromide), instead of AgOTf, in refluxing CH₃CN; the alkylated product 66 was obtained in 74% yield, with 15% of the unreacted starting material 63 being recovered. After optimizing the reaction conditions with compound 63 and bromide 54, a series of S-alkylated sulfonium-ions (67-69) was synthesized analogously in 65-79% yield using alkyl bromides 53, 55, and 56 (Scheme 16).

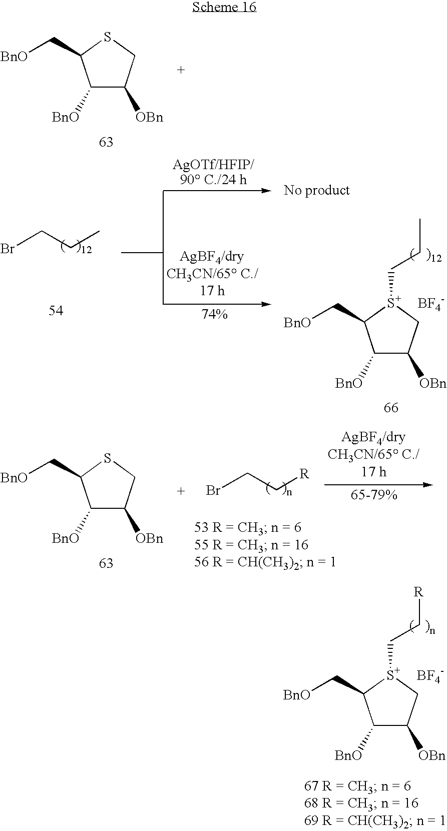

It was also observed that the alkyl iodides 57 and 58 reacted with compound 63 smoothly to produce the corresponding S-alkylated sulfonium-ions 70 and 71 in 90% and 95% yield, respectively. Similarly, the sulfonium-ions 72 and 73 with alkoxy substitutions at the end of the alkyl chain were synthesized in 90% and 89% yield, respectively, using the corresponding iodides 60 and 62 as the alkylating agents (Scheme 17).

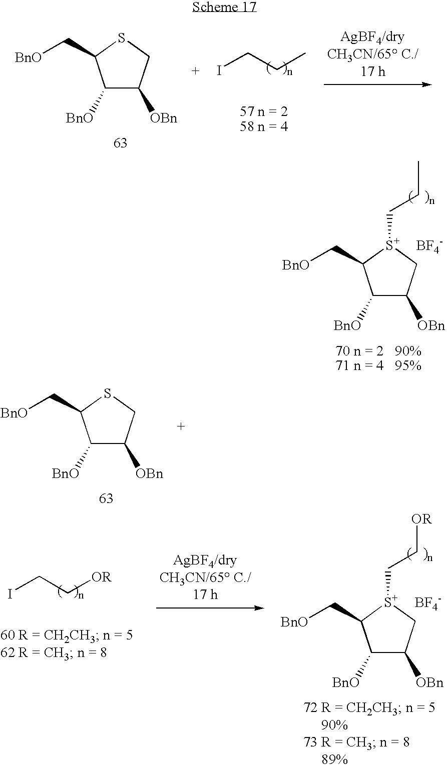

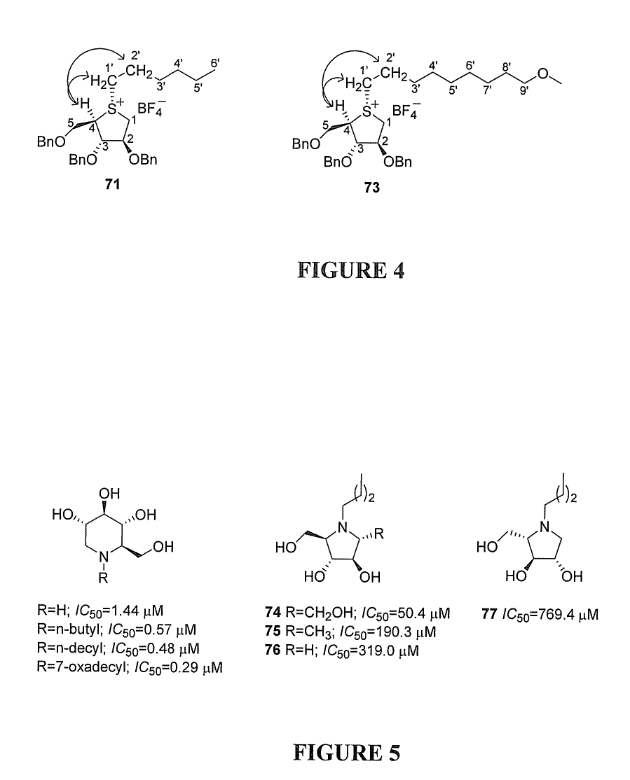

The alkylation reactions proceeded stereoselectively and the inventors did not observe (by TLC) the formation of the diastereomers at the stereogenic sulfur atom in any detectable amounts; the purified products were found to be only one isomer, as indicated by ¹H NMR spectroscopy. The absolute stereochemistry at the stereogenic sulfur center in 71 and 73 was established by means of 1D-NOESY experiments (FIG. 4). Correlation between H-1′ and H-4 and also a correlation between H-2′ and H-4 confirmed the anti relationship between the alkyl side chain and the C-4 substituent on the anhydroarabinitol moiety in both of these compounds (FIG. 4). The stereochemistry at the stereogenic sulfonium center in sulfonium-ions 65-70 and 72 was also assigned to have an anti relationship between the alkyl side chain and the C-4 substituent on the anhydroarabinitol moiety, by analogy with compounds 71 and 73, and based on the inventors' previous, extensive work with alkylation of anhydrothio-D-arabinitol derivatives.

Initial attempts to remove the benzyl protecting groups on the S-alkylated sulfonium ions using 10% Pd/C in MeOH and also in a 1:1 mixture of AcOH/MeOH were not successful. The benzyl groups of 2,3,5-tri-O-benzylsulfonium-ions (65-73) were therefore removed by treatment with boron trichloride at −78° C. in CH₂Cl₂. During the course of deprotection, some of the tetrafluoroborate counterion was exchanged with chloride ion. Similar results were also observed in previous work by the inventors.⁵⁴ Hence, in the cases of sulfonium-ions 66-73, after removal of the benzyl groups, the products were subsequently treated with Amberlyst A-26 resin (chloride form) to completely exchange the tetrafluoroborate counterion with chloride ion to give compounds 45-52, respectively (Scheme 18). In the case of compound 65, the deprotected sulfonium ion 44 was obtained without any counterion exchange, probably due to the shorter reaction time.

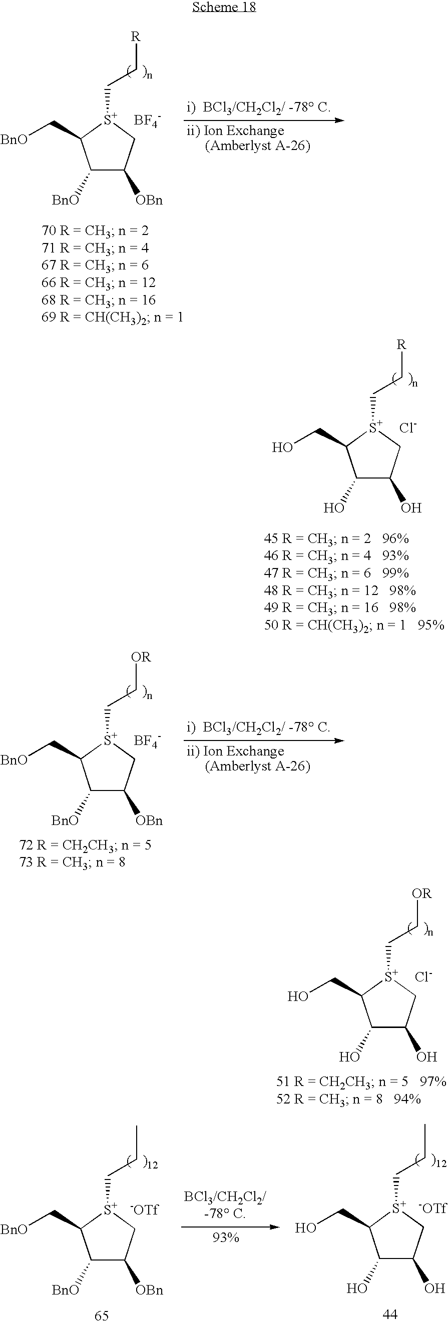

The inhibitory properties of target compounds 44-52 were tested against recombinant human maltase glucoamylase (MGA). All of the synthesized compounds were inhibitors of MGA with IC₅₀ values ranging from 15 to 100 μM (Table 2). Increases in the alkyl chain lengths have significant, albeit not pronounced, effects on the glycosidase inhibitory properties of these compounds. There is a 6.7-fold increase in inhibitory activity upon extension of the alkyl chain from four carbons (45) to eighteen carbons (49). However, these compounds are less active than salacinol which was previously shown to have a Kᵢ value of 0.19±0.02 μM against MGA.⁵⁵

TABLE 2
Experimentally determined IC₅₀ values.^{a}

| Inhibitor | Alkyl chain | IC₅₀ (μM) |
|---|---|---|
| 44 | tetradecyl | 25 |
| 45 | butyl | 100 |
| 46 | hexyl | 50 |
| 47 | octyl | 70 |
| 48 | tetradecyl | 30 |
| 49 | octadecyl | 15 |
| 50 | 2-methylbutyl | 50 |
| 51 | 1-ethoxyhexyl | 100 |
| 52 | 1-methoxynonyl | 100 |
| salacinol | — | 0.19 ± 0.02^{b} |

^{a}Analysis of MGA inhibition was performed using p-nitrophenyl α-D-glucopyranoside as the substrate.
^{b}Reference 55.

The IC₅₀ values for the sulfonium-ions 44-52 against MGA are lower than those previously reported for the N-alkylated imino sugars 75-77 (FIG. 5) against porcine liver α-glucosidase I, but are greater than those for the imino sugars.³³
4.0 Chain-Modified Analogue with Frame Shift of Sulfate Moiety

In a further embodiment of the invention, chain-extended and chain-modified analogues 78-81 of salacinol and blintol were synthestized where the sulfate moiety was shifted to the C-4′ location.

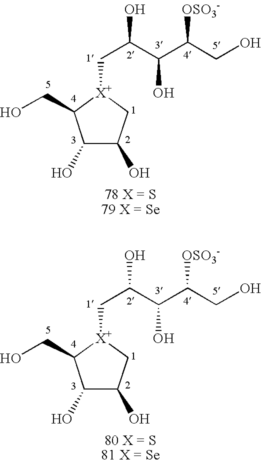

Retrosynthetic analysis revealed that the desired analogues could be synthesized by alkylation of a protected anhydro-D-heteroarabinitol moiety at the ring heteroatom by either an open chain electrophile or a cyclic sulfate derivative, whereby selective attack of the heteroatom at the least hindered primary centre would afford the desired products (Scheme 19).

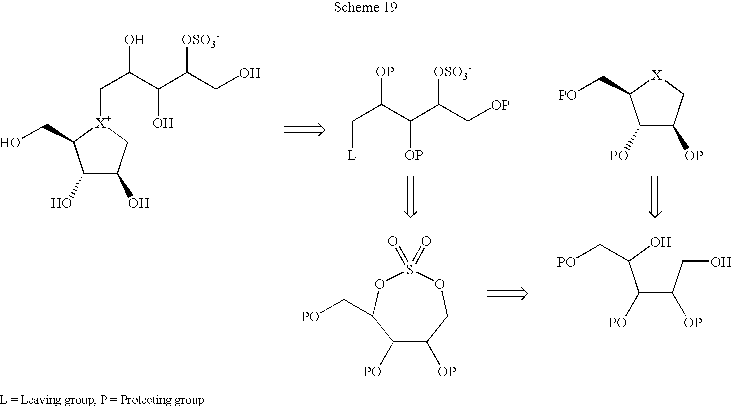

With reference to Scheme 20, the seven-membered cyclic sulfates (86, 88), chosen as the alkylating agents, were synthesized from the corresponding diols. The diols were synthesized in turn from D- or L-xylose in six steps according to the procedure developed for the synthesis of the PMB-protected anhydro-D-selenoarabinitol 85 (Scheme 20).⁵⁶ Thus, diol 82 was used as a key intermediate to synthesize the thioether 84,⁵⁷ the selenoether 85, and the cyclic sulfate 86.

Treatment of the diol 82 with methanesulfonyl chloride in pyridine afforded the dimesylate 83, which on treatment with Na₂S. 9H₂O produced the thioether 84; treatment of 83 with Se/NaBH₄ produced the selenoether 15 (Scheme 20). Treatment of the diol with thionyl chloride and 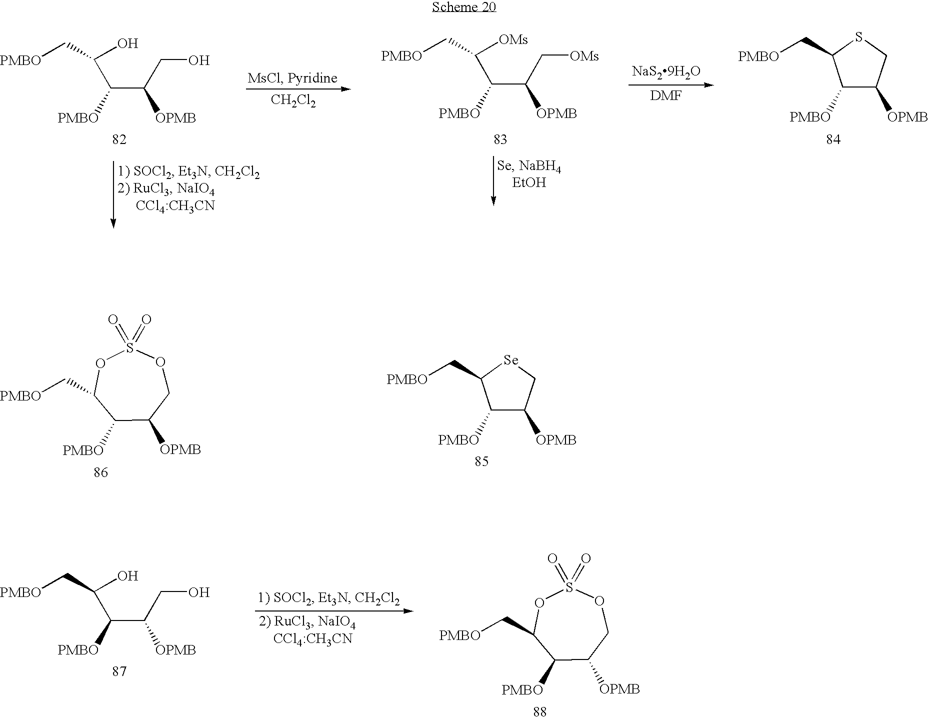
triethylamine gave the cyclic sulfite, which was subsequently oxidized with sodium periodate and ruthenium (III) chloride as a catalyst to afford the cyclic sulfate 86. The corresponding isomer 88 was synthesized in an analogous manner from the enantiomeric diol 87 (Scheme 20), which was obtained in turn from D-xylose.

The alkylation of 84 and 85 with the cyclic sulfates 86 and 88 was examined next (Scheme 21). Thus, the reaction of the thioarabinitol 84 with the cyclic sulfate 86 was found to proceed very slowly at 70° C. in 1,1,1,3,3,3-hexafluoroisopropanol (HFIP) and was terminated before complete consumption of starting materials as we observed that longer reaction times led to decomposition. The coupled product was obtained as the sole product and immediate subsequent removal of the PMB groups by treatment with trifluoroacetic acid then afforded the target compound 78. The stereochemistry at the stereogenic sulfonium center was assigned by means of a NOESY experiment which indicated the presence of the isomer with an anti relationship between C-5 and C-1′. The proton and carbon signals in the ¹H and ¹³C-NMR spectra of 78 were completely assigned with the help of ¹H-¹H COSY, HMQC and HMBC experiments.

Analogously, the reaction of the selenoether 85 with the cyclic sulfate 86 in HFIP gave a coupled product, which was shown to be a 10:1 mixture of diastereomers at the stereogenic selenium center; the major diastereomer was isolated by flash chromatography. Removal of the PMB groups afforded the desired selenonium salt 79. The stereochemistry at the stereogenic selenium center was also assigned as being anti with respect to C₅. In a similar maimer, reaction of the enantiomeric cyclic sulfate 88 with the thio-84 and seleno-85 ethers produced the coupled products in 53% and 66% yield, respectively. Subsequent removal of the PMB groups using TFA yielded the desired products 80 and 81.

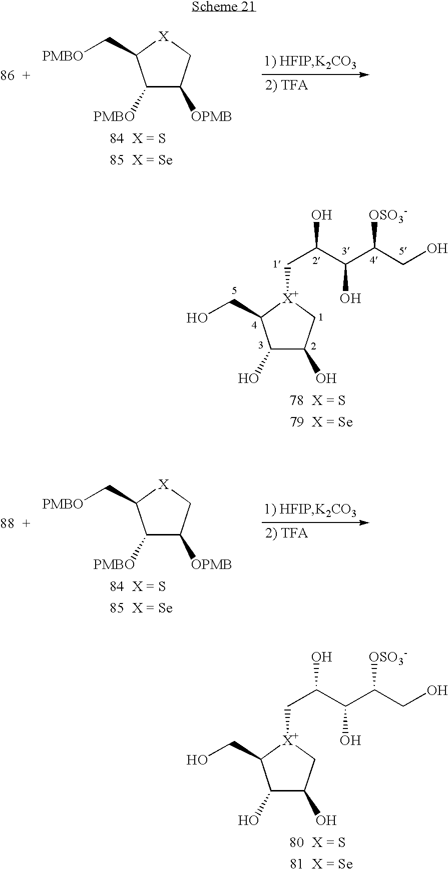

The inhibitory activities of compounds 78-81 against recombinant human MGA was determined. Compounds 78 and 79, with the same configuration at the stereogenic centers in the acyclic chain, have Ki values of 20±4 and 53±5 μM, respectively. These compounds are less active than salacinol and blintol, with Ki values of 0.19±0.02 and 0.49±0.05, respectively.⁵⁵ Interestingly, the analogue 89 (shown below), in which the sulfate moiety is located at the C-3′ and not the C-4′ position, is inactive.⁵⁸ A second 5-carbon chain-extended compound 90 (shown below), with the same configuration at C-3′ and C-4′ as 80, but with the opposite configuration at C-2′ and in which the sulfate moiety is located at the C-3′ and not the C-4′ position, has a Ki value of 0.17±0.03. In contrast, the analogues 80 and 81 are not active. Tanabe et al.⁵⁹ have very recently reported the synthesis of de-O-sulfated analogues of salacinol with monomethyl sulfate and chloride as external counter anions, and these analogues had almost equal inhibitory activities to salacinol against intestinal α-glucosidase in vitro. Further rationalization of these data will have to await knowledge of the detailed contacts between the ligands and the active site from X-ray crystal structures of MGA complexes with candidate inhibitors.

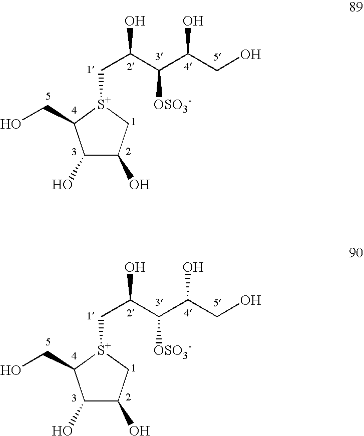

In summary, chain-modified analogues of both salacinol and blintol were synthesized using seven-membered cyclic sulfates as alkylating agents. The cyclic sulfates were prepared from D- and L-xylose. Compounds 78 and 79 showed inhibition of recombinant human MGA with Ki values of 20±4 and 53±5 μM.

### 5.0 EXAMPLES

The following examples will further illustrate the invention in greater detail although it will be appreciated that the invention is not limited to the specific examples.

5.1 Carboxylate Analogues

5.1.1 Sulfonium Carboxylate Analogue

General. Optical rotations were measured at 23° C. Analytical thin-layer chromatography (TLC) was performed on aluminum plates precoated with Merck silica gel 60F-254 as the adsorbent. The developed plates were air-dried, exposed to UV light and/or sprayed with a solution containing 1% Ce(SO₄)₂ and 1.5% molybdic acid in 10% aq H₂SO₄, and heated. Compounds were purified by flash chromatography on Kieselgel 60 (230-400 mesh). ¹H and ¹³C NMR spectra were recorded on the following: Bruker AMX-400 NMR spectrometer at 400.13 MHz, Bruker AMX-600 NMR spectrometer at 600.13 MHz, and Varian INOVA 500 NMR spectrometer at 499.97 MHz for ¹H. Chemical shifts are given in ppm downfield from TMS for those measured in CDCl₃ and CD₃OD and from 2,2-dimethyl-2-silapentane-5-sulfonate (DSS) for those spectra measured in D₂O. Chemical shifts and coupling constants were obtained from a first-order analysis of the spectra. Assignments were fully supported by two-dimensional ¹H,¹H (COSY), and ¹H,¹³C (HMQC) experiments using standard Bruker or Varian pulse programs. Processing of the spectra was performed with standard UXNMR and WINNMR software (Bruker) or MestReC software (Varian). MALDI mass spectra were obtained on a PerSeptive Biosystems, Voyager DE time-of-flight spectrometer for samples dispersed in a 2,5-dihydroxybenzoic acid matrix.

2,3-O-Isopropylidene-D-ribonolactone (13).³⁹ To a suspension of D-ribonolactone 12 (10 g, 68 mmol) in actone (200 mL) was added concentrated sulfuric acid (4 mL) dropwise while the solution was cooled in an ice bath. The starting material dissolved in 5 minutes. The mixture was stirred for 12 h at room temperature. Ammonia gas was passed through the ice-cooled solution. The resulting white solid was filtered off and the filtrate was concentrated under reduced pressure. The crude product was purified by column chromatography (Hexanes-EtOAc, 1:3) to afford 13 (10.6 g, 80%) as a white solid: mp 134-137° C.; lit. mp 135-138.

2,3-O-Isopropylidene-5-O-tosyl-D-ribonolactone (14).⁴⁰ To a solution of compound 13 (1.0 g, 5.3 mmol) in CH₂Cl₂ (25 mL) was added Et₃N (0.59 g, 1.1 eqiv.), and a catalytic amount of 4-(dimethylamino)pyridine. After 5 minutes, tosyl chloride (1.2 g, 1.2 eqiv.) was added in portions at 0° C. and the resulting mixture was stirred at the room temperature for 16 h. The reaction mixture was poured into water (40 mL) and CH₂Cl₂ (30 mL). The organic phase was dried (Na₂SO₄) and concentrated on a rotary evaporator. The product was purified by flash chromatography (Hexanes-EtOAc, 3:1) to afford 14 (1.6 g, 88%) as a white solid. ¹H NMR (CDCl₃): δ 7.77-7.37 (4H, 2d, J_{A,B}=8.2 Hz, Ar), 4.77 (1H, d, J₂₃=5.6 Hz, H-2), 4.75 (1H, d, H-3), 4.68 (1H, dd, H-4), 4.34 (1H, dd, J_{4,5b}=1.9 Hz, J_{5a,5b}=11.2 Hz, H-5b), 4.18 (1H, dd, J_{4,5a}=2.5 Hz, H-5a), 2.47 (3H, s, CH₃Ph), 1.46 and 1.39 (6H, 2s, C(CH₃)₂).

4,5-Anhydro-2,3-O-isopropylidene-D-ribonic acid benzyl ester (15).⁴⁰ A solution of sodium benzylate prepared from benzyl alcohol (0.71 g, 6.57 mmol) and NaH (60 nmg, 1.5 mmol) in DMF (7.1 mL) was added to compound 14 (0.5 g, 1.46 mmol) in DMF (1.1 mL) at 0° C. The reaction mixture was stirred for 1 h. Solvent was removed under high vacuum. The white solid was filtered off and the filtrate was concentrated. The crude product was purified by column chromatography (Hexanes-EtOAc, 5:1) to afford 15 (350 mg, 86%) as a colorless oil. ¹H NMR (CD₂Cl₂): δ 7.42-7.37 (5H, m, Ar), 5.22 and 5.17 (2H, 2d, J_{A,B}=12.1 Hz, CH₂Ph), 4.80 (1H, d, J_{2,3}=6.92 Hz, H-2), 4.11 (1H, dd, H-3), 2.93 (1H, ddd, J_{3,4}=6.19 Hz, J_{4,5a}=3.90 Hz, J_{4,5b}=2.55 Hz, H-4), 2.64 (1H, dd, J_{5a,5b}=5.21 Hz, H-5b), 2.61 (1H, dd, H-5a), 1.58 and 1.39 (6H, 2s, C(CH₃)₂).

1,4-Dideoxy-1,4-[benyl-[(2′S,3′S,4′R)-2′,3′-O-isopropylidene-2′,3′,4′-trihydroxy-4′-carboxybutylate]-episulfoniumyl-idene]-L-arabinitol salt (16). A mixture of compound 15 (130 mg, 0.47 mmol) and compound 8 (196 mg, 1.0 equiv) was dissolved in dry CH₂Cl₂ (2 mL) and CF₃CO₂H (53 mg, 1.0 equiv) was added. The mixture was stirred at room temperature for 3 h. The solvent was removed under reduced pressure, and column chromatography (EtOAc-MeOH—H₂O, 40:1:1) of the crude product gave a colorless oil (190 mg, 58%). MALDI-TOF MS: m/e 699.42 (M⁺).

1,4-Dideoxy-1,4-[benyl-[(2′S,3′S,4′R)-2′,3′,4′-trihydroxy-4′-carboxybutylate]-episu-Ifoniumyl-idene]-L-arabinitol chloride (17). Without further purification, the compound 16 (250 mg, 0.36 mmol) was dissolved in a mixture of concentrated HCl (1 mL) and methanol (40 mL). The mixture was stirred at rt for 6 h. The solvent was removed under reduced pressure and column chromatography (CH₂Cl₂-MeOH, 8:1) of the crude product gave 17 as a colorless oil (168 mg, 71%). [α]_{D} +20° (c 1.0, CH₂Cl₂). ¹H NMR (CH₂Cl₂): δ 7.43-7.35 (20H, m, Ar), δ 5.07 (2H, 2d, J_{A,B}=11.1 Hz, CO₂CH₂Ph), 4.51-4.31 (6H, m, 3OCH₂Ph), 4.49 (1H, m, H-2), 4.45 (1H, m, H-2′), 4.42 (1H, m, H-4′), 4.21 (1H, m, H-3), 4.18 (1H, m, H-4), 4.13 (1H, m, H-1b), 4.07 (1H, m, H-3′), 4.06 (1H, m, H-1′b), 3.96 (1H, m, H-1a), 3.32 (1H, br, H-1′a), 3.28 (1H, m, H-5b), 3.23 (1H, m, H-5a). ¹³C NMR (CH₂Cl₂): δ 173.54 (C-5′), 139.13, 138.39, 138.35, 137.84 (4Cᵢₚₛₒ), 130.57-129.52 (20C_{Ar}), 84.88 (C-3), 84.82 (C-2), 77.12 (C-3′), 75.34, 74.11, 73.87 (3OCH₂Ph), 75.06 (C-4′), 69.06 (C-5), 68.89 (C-2′), 68.58 (CO₂CH₂Ph), 67.04 (C-4), 53.74 (C-1′), 49.57 (C-1). MALDI-TOF MS: m/e 659.20 (M⁺). Anal. calcd. For C₃₈H₄₃ClO₈S: C, 65.65; H, 6.23. found: C, 65.62; H, 6.40.

1,4-Dideoxy-1,4-[[(2′S,3′S,4′R)-2′,3′,4′-trihydroxy-4′-carboxybutyl]-episulfoniumyl-idene]-L-arabinitol inner salt (4). Without further purification, compound 16 (100 mg, 0.14 mmol) was dissolved in CH₂Cl₂ (5 mL). BCl₃ was passed through the solution for 2 minutes at −78° C. The solution was stirred at −78° C. for 1 h. Air was passed through the reaction flask until no white gas formed. H₂O was added slowly to quench the reaction. The resulting mixture was concentrated under reduced pressure. Column chromatography (7:3:1 EtOAc-MeOH—H₂O and then pure H₂O) of the crude product gave 4 (21 mg, 50%). [α]_{D} +29° (c 0.2, H₂O). ¹H NMR (D₂O): 54.68 (1H, dt, J_{1,2}=3.7 Hz, H-2), 4.40 (1H, t, J_{2,3}=J_{3,4}=3.3 Hz, H-3), 4.22 (1H, b, H-2′), 4.14 (1H, d, J_{3′,4′}=2.7 Hz, H-4′), 4.08 (1H, ddd, H-4), 4.03 (1H, dd, J_{4,5b}=5.2 Hz, J_{5a,5b}=12.3 Hz, H-5b), 3.95 (1H, b, H-3′), 3.91 (1H, dd, J_{4,5a}=8.1 Hz, H-5a), 3.82 (2H, m, H-1′), 3.78 (2H, d, H-1). ¹³C NMR (D₂O): δ 179.81 (C-5′), 79.87 (C-3), 79.01 (C-2), 76.89 (C-3′), 75.55 (C-4′), 71.99 (C-4), 69.55 (C-2′), 61.38 (C-5), 51.80 (C-1′), 49.17 (C-1). MALDI-TOF MS: m/e 321.35 (M⁺+Na), 299.48 (M⁺+H). HR-MS calcd. for C₁₀H₁₉O₈S (M+H): 299.0801. found: 299.0801.

1,4-Dideoxy-1,4-[benyl-[(2′S,3′S,4′R)-2′,3′-O-isopropylidene-2′,3′,4′-trihydroxy-4′-carboxybutylate]-episulfoniumyl-idene]-D-arabinitol salt (18). A mixture of compound 15 (277 mg, 1 mmol) and compound 11 (417 mg, 1.0 equiv) was dissolved in dry CH₂Cl₂ (2 mL) and CF₃CO₂H (113 mg, 1.0 equiv) was added. The mixture was stirred at room temperature for 3 h. The solvent was removed under reduced pressure, and column chromatography (EtOAc-MeOH—H₂O, 40:1:1) of the crude product gave a colorless oil (460 mg, 66%). MALDI-TOF MS: m/e 699.46 (M⁺).

1,4-Dideoxy-1,4-[benyl-[(2′ S,3′S,4′R)-2′,3′,4′-trihydroxy-4′-carboxybutylate]-episu-lfoniumyl-idene]-D-arabinitol chloride (19). Without further purification, the compound 18 (200 mg, 0.23 mmol) was dissolved in a mixture of concentrated HCl (1 mL) and methanol (40 mL). The mixture was stirred at rt for 6 h. The solvent was removed under reduced pressure and column chromatography (CH₂Cl₂-MeOH, 8:1) of the crude product gave 19 as a colorless oil (142 mg, 75%). [a]_{D} +15° (c 0.9, CH₂Cl₂). ¹H NMR (CD₃OD): δ 7.40-7.20 (20H, m, Ar), 5.17 (2H, s, CO₂CH₂Ph), 4.67-4.44 (6H, m, 3OCH₂Ph), 4.63 (1H, d, H-2), 4.43 (1H, s, H-3), 4.35 (1H, d, J_{3′,4′}=3.5 Hz, H-4′), 4.30 (1H, dd, J_{4,5b}=6.7 Hz, J_{4,5a}=9.6 Hz, H-4), 4.24 (1H, ddd, J_{1′a,2′}=7.2 Hz, J_{1′b,2′}=3.4 Hz, H-2′), 4.02 (1H, d, J_{1a,1b}=13.1 Hz, H-1b), 3.96 (1H, dd, J_{2′,3′}=6.3 Hz, H-3′), 3.84 (1H, dd, J_{1′a,1′b}=13.0 Hz, H-1′b), 3.80 (1H, dd, H-5b), 3.79 (1H, dd, J_{1a,2}=3.1 Hz, H-1a), 3.73 (1H, dd, H-1′a), 3.70 (1H, dd, J_{5a,5b}=10.0 Hz, H-5a). ¹³C NMR (CD₃OD): δ 173.40 (C-5′), 138.56-138.02 (4Cᵢₚₛₒ), 129.98-129.19 (20C_{Ar}), 84.45 (C-2), 84.11 (C-3), 76.30 (C-3′), 74.46 (C-4′), 73.68, 73.31, 73.15 (3OCH₂Ph), 68.55 (C-2′), 68.02 (C-5), 67.88 (CO₂CH₂Ph), 67.66 (C-4), 51.84 (C-1′), 49.81 (C-1). MALDI-TOF MS: m/e 659.25 (M⁺). Anal. calcd. For C₃₉H₄₃F₃O₁₁S₂¹: C, 57.91; H, 5.36. found: C, 57.55; H, 5.49. ≠¹ Counterion was exchanged to triflate.

1,4-Dideoxy-1,4-[[(2′S,3′S,4′R)-2′,3′,4′-trihydroxy-4′-carboxybutyl]-episulfoniumyl-idene]-D-arabinitol inner salt (5). Without further purification, compound 18 (150 mg, 0.21 mmol) was dissolved in CH₂Cl₂ (10 mL). BCl₃ was passed through the solution for 2 minutes at −78° C. The solution was stirred at −78° C. for 1 h. Air was passed through the reaction flask until no white gas formed. H₂O was added slowly to quench the reaction. The resulting mixture was concentrated under reduced pressure. Column chromatography (7:3:1 EtOAc-MeOH—H₂O and then pure H₂O) of the crude product gave 5 (33 mg, 52%). [α]_{D} −12° (c 0.6, H₂O). ¹H NMR (D₂O): δ 4.57 (1H, dt, J_{1,2}=3.8 Hz, H-2), 4.26 (1H, dd, J_{2,3}=3.5 Hz, H-3), 4.09 (1H, ddd, J_{1′a,2′}=8.8 Hz, J_{1b,2′}=3.1 Hz, J_{2′,3′}=5.5 Hz, H-2′), 4.01 (1H, d, J_{3′,4′}=3.7 Hz, H-4′), 3.96 (1H, dd, J_{4,5b}=4.7 Hz, J_{5a,5b}=12.1 Hz, H-5b), 3.89 (1H, ddd, J_{3,4}=3.2 Hz, J_{4,5a}=8.1 Hz, H-4), 3.86 (1H, dd, H-3′), 3.77 (1H, m, H-5a), 3.75 (1H, dd, J_{1′a,1′b}=13.4 Hz, H-1′b), 3.72 (2H, d, H-1), 3.64 (1H, dd, H-1′a). ¹³C NMR (D₂O): δ 179.47 (C-5′), 79.35 (C-3), 78.65 (C-2), 76.56 (C-3′), 75.14 (C-4′), 71.50 (C-4), 69.14 (C-2′), 61.02 (C-5), 51.50 (C-1′), 49.66 (C-1). HRMS calcd. for C₁₀H₁₉O₈S (M+H): 299.0801. found: 299.0795.

5.1.2 Nitrogen Carboxylate Analogue

Enzyme Activity Assays: Measurement of dGMII inhibition was carried out as outlined in the literature.¹² Analysis of recombinant MGA inhibition and determination of the kinetic constants for competitive inhibition have been described.⁶⁰ Briefly, analysis of MGA inhibition was performed using maltose as the substrate, and measuring the release of glucose. Reactions were carried out in 100 mM MES buffer pH 6.5 at 37° C. The reaction was stopped by boiling for 3 min. 20 μL aliquots were taken and added to 100 μL of glucose oxidase assay reagent (Sigma) in a 96-well plate. Reactions were developed for 1 hour and absorbance was measured at 450 nm to determine the amount of glucose produced by MGA activity in the reaction. All reactions were performed in triplicate and absorbance measurements were averaged to give a final result.

Enzyme Kinetics: Kinetic parameters of recombinant MGA were determined using the glucose oxidase assay to follow the production of glucose upon addition of enzyme (15 nM) at increasing maltose concentrations (from 2.5 mM to 30 mM) with a reaction time of 15 minutes. The Kᵢ value was determined by measuring the rate of maltose hydrolysis by MGA at varying inhibitor concentrations. Data were plotted in Lineweaver-Burk plots (1/rate vs. 1/[substrate]) and the Kᵢ value was determined by the equation Kᵢ=Kₘ[I]/(Vₘₐₓ)s−Kₘ, where “s” is the slope of the line. The Kᵢ reported was an average of the Kᵢ values obtained from each of the different inhibitor concentrations.

Structure Determination of dGMII:16 complex. Preparation of dGMII crystals soaked with 21 was carried out essentially as described in the literature.¹² In this case however, the crystals were first washed with reservoir buffer containing phosphate instead of Tris, to reduce any effects of Tris binding in the active site. The crystals were soaked for 24 hours with a 2 mM solution of 21 in phosphate containing reservoir buffer. The crystals were passed through phosphate buffered cryo-solutions containing 1 mM inhibitor prior to rapid freezing in a liquid nitrogen stream. X-ray diffraction data was collected at 100 K with a Bruker X8 Proteum system consisting of a CCD detector and a Bruker Microstar rotating anode generator. Data were integrated and scaled using the Proteum suite of programs (Bruker AXS, Madison Wis.). Structure solution and refinement were carried out using the programs CNS⁶¹ and O⁶² as previously described.¹² Diagrams were rendered in Pymol.⁶³

N-Allyl-2,3,5-tri-O-benzyl-1,4-(dideoxy-1,4-imino-L-arabinitol (27), N-allyl-2,3,5-tri-O-benzyl-1,4-dideoxy-1,4-imino-D-arabinitol (30), 2,3,5-tri-O-benzyl-1,4-dideoxy-1,4-imino-L-arabinitol (28), 2,3,5-tri-O-benzyl-1,4-dideoxy-1,4-imino-D-arabinitol (31) were synthesized according to the original literature procedures.^{6,44,45}

6′-((2,3,5-Tri-O-benzyl-1,4-dideoxy-1,4-imino-L-arabinitol)-4-N-yl)-2′,3′-di-O-benzyl-6′-deoxy-L-ascorbic acid (32). A mixture of 2,3,5-tri-O-benzyl-1,4-dideoxy-1,4-imino-L-arabinitol 28 (404 mg, 1.0 mmol) and 5,6-anhydro-2,3-di-O-benzyl-L-ascorbic acid 25 (340 mg, 1.0 equiv) were dissolved in dry CH₃CN (5 mL). The mixture was stirred in a round bottom flask in an oil-bath (70° C.) overnight. The solvent was removed under reduced pressure, and the product was purified by column chromatography (Hexanes-EtOAc, 3:1) to afford 32 (612 mg, 82%) as a yellow oil. [α]_{D} +23° (c 0.6, CH₂Cl₂). ¹H NMR (CDCl₃): δ 7.11-7.30 (25H, m, Ar), 5.12 and 5.04 (2H, 2d, J_{A,B}=11.8 Hz, C═C—OCH₂Ph), 5.01 and 4.98 (2H, 2d, J_{A,B}=11.3 Hz, C═C—OCH₂Ph), 4.57 (1H, d, J_{2′,3′}=1.5 Hz, H-3′), 4.44 and 4.42 (2H, 2d, J_{A,B}=6.3 Hz, CH₂Ph), 4.39 and 4.36 (2H, 2d, J_{A,B}=9.4 Hz, CH₂Ph), 4.35 and 4.33 (2H, 2d, J_{A,B}=12.0 Hz, CH₂Ph), 3.89 (1H, ddd, J_{1a,2}=1.6 Hz, J_{1b,2}=5.5 Hz, J_{2,3}=1.8 Hz, H-2), 3.84 (1H, ddd, J_{1′a,2′}=8.0 Hz, J_{1′b,2′}=6.0 Hz, H-2′), 3.76 (1H, dd, J_{3,4}=3.9 Hz, H-3), 3.46 (1H, dd, J_{4,5a}=5.9 Hz, J_{5a,5b}=9.8 Hz, H-5a), 3.43 (1H, dd, J_{4,5b}=5.9 Hz, H-5b), 3.09 (1H, dd, J_{1a,1b}=10.5 Hz, H-1a), 2.88 (1H, dd, J_{1′a,1′b,}=13.0 Hz, H-1′a), 2.84 (1H, dt, H-4), 2.77 (1H, dd, H-1b), 2.73 (1H, dd, H-1′b). ¹³C NMR (CDCl₃): δ 169.57 (C-6′), 157.44 (C-4′), 137.86, 137.79, 137.73, 135.85, 135.32 (5Cᵢₚₛₒ), 128.76-127.24 (25C_{Ar}), 120.68 (C-5′), 84.59 (C-3), 81.63 (C-2), 75.54 (C-3′), 73.60 and 72.98 (2C═C—OCH₂Ph), 72.87, 71.09, 70.78 (3CH₂Ph), 70.34 (C-5), 68.95 (C-4), 66.93 (C-2′), 58.00 (C-1), 56.69 (C-1′). MALDI-TOF MS: m/e 763.85 (M⁺+Na), 742.075 (M⁺+H). Anal. calcd. For C₄₆H₄₇O₈N: C, 74.43; H, 6.38; N, 1.89. found: C, 74.20; H, 6.35; N, 2.14.

6′-((1,4-Dideoxy-1,4-imino-L-arabinitol)-4-N-ammonium)-6′-deoxy-L-gulonate (20). The protected compound 32 (300 mg, 0.4 mmol) was dissolved in AcOH—H₂O (4:1, 6 mL) and stirred with Pd/C (30 mg) under H₂ (70 psi). After 4 days, the reaction mixture was filtered thlough a cotton, which was subsequently washed with H₂O. The combined filtrates were concentrated under vacuum. Concentrated hydrochloric acid (1 mL) was added and the mixture was concentrated by high vacuum. The obtained solid was dissolved in aqueous K₂CO₃ solution (1 mL, pH=9.0) and the mixture was stirred for 3 h. The solution was neutralized with dilute hydrochloric acid and then concentrated. The residue was purified by Sephadex G-10 chromatography column to give 20 as an amorphous solid (104 mg, 78%). [α]_{D} +100 (c 0.1, H₂O). ¹H NMR (D₂O, pH=12.0): δ 4.03 (1H, d, J_{4′,5′}=4.5 Hz, H-5′), 3.98 (1H, brd, H-2), 3.83 (1H, d, H-4′), 3.79 (2H, brd, H-3, H-2′), 3.64 (1H, m, H-3′), 3.61 (2H, m, 2H-5), 2.99 (1H, d, J_{1a,1b}=11.1 Hz, H-1a), 2.91 (1H, dd, J_{1′a,1′b}=12.8 Hz, J_{1′a,2′}=5.0 Hz, H-1′a), 2.73 (1H, dd, J_{1b,2}=5.0 Hz, H-1b), 2.47 (1H, m, H-4), 2.43 (1H, dd, J_{1′b,2}=7.1 Hz, H-1′b). ¹³C NMR (D₂O, pH=12.0): δ 179.13 (C-6′), 76.50 (C-3), 73.35 (C-2), 71.45 (C-5′), 70.27 (C-4′), 69.86 (C-4), 69.35 (C-3′), 68.39 (C-2′), 58.46 (C-5), 57.44 (C-1), 54.98 (C-1′). MALDI-TOF MS: m/e 334.47 (M⁺+Na), 312.43 (M⁺+H). Anal. calcd. For C₁₁H₂₁O₉N: C, 42.44; H, 6.80; N, 4.50. found: C, 42.19; H, 6.66; N, 4.36.

6′-((2,3,5-Tri-O-benzyl-1,4-dideoxy-1,4-imino-D-arabinitol)-4-N-yl)-2′,3′-di-O-benzyl-6′-deoxy-L-ascorbic acid (34). A mixture of 2,3,5-tri-O-benzyl-1,4-dideoxy-1,4-imino-D-arabinitol 31 (444 mg, 1.1 mmol) and 5,6-anhydro-2,3-di-O-benzyl-L-ascorbic acid 25 (374mg, 1.0 equiv) were dissolved in dry CH₃CN (5 mL). The mixture was stirred in a round bottom flask in an oil-bath (70° C.) overnight. The solvent was removed under reduced pressure, and column clnomatography (Hexanes-EtOAc, 3:1) of the crude product gave 34 (605 mg, 74%) as a yellow oil. [α]_{D} +6° (c 0.7, CH₂Cl₂). ¹H NMR (CDCl₃): δ 7.13-7.30 (25H, m, Ar), 5.22 and 5.04 (2H, 2d, J_{A,B}=11.7 Hz, C═C—OCH₂Ph), 5.11 and 5.05 (2H, 2d, J_{A,B}=11.3 Hz, C═C—OCH₂Ph), 4.51 (1H, brd, H-3′), 4.50-4.40 (6H, m, 3CH₂Ph), 3.96 (1H, dt, J_{1a,2}=1.7 Hz, J_{1b,2}=5.5 Hz, H-2), 3.88 (1H, dd, J_{2,3}=1.8 Hz , J_{3,4}=4.4 Hz, H-3), 3.87 (1H, m, H-2′), 3.53 (1H, dd, J_{4,5a}=5.3 Hz, J_{5a,5b}=9.8 Hz, H-5a), 3.51 (1H, dd, J_{4,5b}=5.6 Hz, H-5b), 3.23 (1H, d, J_{1a,1b}=10.4 Hz, H-1a), 3.17 (1H, dd, J_{1′a,1′b,}=12.5 Hz, J_{1′a,2′,}=10.9 Hz, H-1′a), 2.88 (1H, q, H-4), 2.64 (1H, dd, H-1b), 2.52 (1H, dd, J_{1′b,2′,}=3.5 Hz, H-1′b). ¹³C NMR (CDCl₃): δ 169.90 (C-6′), 157.43 (C-4′), 138.24, 138.21, 138.19, 136.36, 135.80 (5Cᵢₚₛₒ), 128.96-127.64 (25C_{Ar}), 121.22 (C-5′), 84.83 (C-3), 82.03 (C-2), 75.94 (C-3′), 74.20 and 73.62 (2C═C—OCH₂Ph), 73.49, 71.79, 71.38 (3CH₂Ph), 70.28 (C-5), 69.12 (C-4), 66.28 (C-2′), 57.40 (C-1), 57.10 (C-1′). MALDI-TOF MS: m/e 764.03 (M⁺+Na), 741.95 (M⁺+H). Anal. calcd. For C₄₆H₄₇O₈N: C, 74.43; H, 6.38; N, 1.89. found: C, 74.27; H, 6.39; N, 2.03.

6′-((1,4-Dideoxy-1,4-imino-D-arabinitol)-4-N-ammonium)-6′-deoxy-L-gulonic acid hydro-chloride (21). The protected compound 34 (600 mg, 0.8 mmol) was dissolved in AcOH—H₂O (4:1, 10 mL) and stirred with Pd/C (50 mg) under H₂ (70 psi). After 4 days, the reaction mixture was filtered through cotton, which was subsequently washed with H₂O. The combined filtrates were concentrated under vacuum. Concentrated hydrochloric acid (2 mL) was added and the mixture was concentrated by high vacuum. The obtained solid was dissolved in aqueous NaOH solution (2 mL, pH=9.0) and the mixture was stirred for 3 h. The solution was neutralized with dilute hydrochloric acid. Na⁺ ion was removed with excess Amberlite IR-120-P (H⁺ form) and the resin was removed by filtration. The aqueous solution was concentrated to give 21 as an amorphous solid (165 mg, 62%). [α]_{D} +2° (c 0.5, H₂O). ¹H NMR (D₂O, pH=12.0): δ 3.94 (1H, d, J_{4′,5′}=4.8 Hz, H-5′), 3.89 (1H, m, H-2), 3.75-3.71 (3H, m, H-4′, H-3, H-2′), 3.57-3.52 (2H, m, 2H-5), 3.50 (1H, m, H-3′), 2.90 (1H, dd, J_{1a,1b}=10.4 Hz, J_{1a,2}=1.1 Hz, H-1a), 2.76 (1H, dd, J_{′a,1′b}=13.0 Hz, J_{1′a,2′}=9.4 Hz, H-1′a), 2.57 (1H, dd, J_{b,2}=5.6 Hz, H-1b), 2.36 (1H, m, H-1′b), 2.33 (1H, m, H-4); ¹³C NMR (D₂O, pH=12.0): δ 179.00 (C-6′), 79.24 (C-3), 75.95 (C-2), 74.18 (C-5′), 72.71 (C-4′), 72.30 (C-4, C-3′), 70.06 (C-2′), 60.70 (C-5), 58.99 (C-1), 57.54 (C-1′). MALDI-TOF MS: m/e 334.52 (M⁺+Na), 312.32 (M⁺+H). Anal. calcd. For C₁₁H₂₂O₉NCl: C, 38.00; H, 6.37; N, 4.03. found: C, 38.15; H, 6.45; N, 3.86.

5.2 Phosphate Analogue

General: Optical rotations were measured at 23° C. ¹H and ¹³C-NMR spectra were recorded on Varian INOVA 500 NMR spectrometer at 500 and 125 MHz, respectively. All assignments were confirmed with the aid of two-dimensional experiments (¹H-¹H gCOSY, gHMQC). Column chromatography was performed with Merck silica gel 60 (230-400 mesh). MALDI-TOF mass spectra were recorded on a PerSeptive Biosystems Voyager-DE spectrometer, using 2,5-dihydroxybenzoic acid as a matrix.

### N-hydroxyethyl-2,3,5-tri-O-benzyl-1,4-dideoxy-1,4-imino-D-arabinitol (39)

Ethanolamine (34.6 mmol, 2.08 mL) was added to a stirred solution of the dimesylate (38) (3.46 mmol, 2.0 g) in acetonitrile (15 mL) and the mixture was heated to reflux for 20 h under N₂. The reaction mixture was concentrated in vacuo and the residue was dissolved in ethyl acetate (30 mL) and washed with water (3×15 mL). The organic layer was dried over anhydrous sodium sulfate and evaporated. Purification by column chromatography (EtOAc:Hexanes, 7:3) gave (39) as a colorless oil (1.28 g 83%). [α]_{D}²³ +1.00 (c=1.1, MeOH). ¹H-NMR (CDCl₃, 500 MHz): δ 7.31 (15H, m, 3×Ph), 4.50 (6H, ddd, J=12.0 Hz, 3×CH₂Ph), 3.99 (1H, dd, J_{2,3}=1.6 Hz, J_{2,1}=3.7 Hz, H-2), 3.89 (1H, bd, J_{3,4}=2.5 Hz, H-3), 3.65-3.52(2×2H, m, H-2′ and 5-H), 3.28(1H, bd, J_{1,2}=3.59 Hz, H-1a), 3.10-3.06 (1H, m, H-1′a), 2.91 (1H, bd, J_{4,5}=3.6 Hz, H-4), 2.69 (1H, dd, J_{1,2}=5.3 Hz, J_{1a,1b}=10.6 Hz, H-1b), 2.61 (1H, d, J_{1′a,1′b}=12.6 Hz, H-1′b). ¹³C-NMR (CDCl₃, 125 MHz): δ 138.39, 138.32, 138.25 (3×Cᵢₚₛₒ), 128.6-127.8 (15C, 3×5, Ph), 85.2(C-3), 82.0(C-2), 71.68, 71.39, 71.04 (3×CH₂Ph), 69.3(C-4), 60.0 (C-2′), 57.5(C-1), 57.3(C-1′). MALDI-TOF-MS: m/e 448.04 (M⁺+H). Anal. Calcd for C₂₈H₃₃NO₄: C, 75.14; H, 7.43; N, 3.13. Found: C, 75.44; H, 7.10; N, 3.40.

### 2′-Phosphorylethyl-2,3,5-tri-O-benzyl-1,4-dideoxy-1,4-imino-D-arabinitol (40)

To a well-stirred mixture of triphenylphosphine (3.35 mmol, 0.9 g), and diisopropyl azodicarboxylate (DIAD) (3.35 mmol, 0.65 mL) in anhydrous THF (5 mL) at 0° C., dibenzyl phosphate (3.35 mmol, 0.94 g) was added. After stirring the reaction mixture for 5 min at 0° C., a solution of N-hydroxyethyl-2,3,5-tri-O-benzyl-1,4-dideoxy-1,4-imino-D-arabinitol (39) (2.24 mmol, 1.0 g) in THF (5 mL) was added dropwise at 0° C. under N₂, and the reaction mixture was stirred for 3 h at room temperature. After completion of the reaction, THF was removed in vacuo and the residue was purified by column chromatography (EtOAc:Hexanes, 4:6) to give compound (40) as a colorless oil (1.2 g, 76%). [α]_{D}²³ +26.0° (c=1.0, MeOH). ¹H-NMR (CDCl₃, 500 MHz): δ 7.25-7.17(25H, m, 5×Ph), 5.07-4.92 (m, 4H, 2×POCH₂Ph), 4.42-4.29 (6H, m, 3×OCH₂Ph), 4.10-3.96(2H, m, H-2′), 3.82(1H, d, J_{2,3}=5.0 Hz, H-2), 3.73 (1H, d, J_{3,4}=3.7 Hz, H-3), 3.42 (2H, ddd, J_{5,4}=6.0 Hz, J_{5a,5b}=9.7 Hz, H-5), 3.16 (1H, d, J_{1a,1b}=10.5 Hz, H-1a), 3.07 (1H, td, J_{1′,2′}=6.3 Hz, J_{1′a,1′b}=12.9 Hz, H-1′a) 2.72 (1H, dd, J_{4,5}=5.7 Hz, H-4), 2.57 (2H, m, H-1b, H-1′b). ¹³C-NMR (CDCl₃, 125 MHz): δ 138.53, 138.36, 138.31, 136.22, 138.18 (5×Cᵢₚₛₒ) 128.7-127.7(5×Ph), 85.3(C-3), 81.8(C-2), 73.4-71.5 (CH₂Ph) 69.44 (d, J_{C,P}=5.4 Hz, POCH₂Ph), 68.1(d, ²J_{C,P}=5.7 Hz, C-2′), 58.0(C-1), 54.84 (d, ³J_{C,P}=6.6 Hz, C-1′). MALDI-TOF-MS: m/e 708.3 (M⁺+H). Anal. Calcd for C₄₂H₄₆NO₇P: C, 71.27; H, 6.55; N, 1.98. Found: C, 71.09; H, 6.50; N, 1.92.

### 2′-Phosphorylethyl-1,4-dideoxy-1,4-imino-D-arabinitol (36)

A solution of compound (40) (1.7 mmol, 1.2 g) in methanol (5 mL) containing 10% palladium on carbon (0.5 g) was stirred under 80 psi of hydrogen at room temperature for 10 h. The mixture was filtered, and the solvent was removed in vacuo to give a white solid. The compound was purified further by column chromatography (MeOH:H₂O, 8:2) to give (36) (0.26 g, 61%). m.p. 178-180° C. [α]_{D}²³ +2.1° (c=1.1, H₂O). ¹H-NMR (D₂O, 500 MHz): δ 4.36 (1H, td, J_{2,3}=2.0 Hz, J_{2,1}=4.3 Hz, H-2), 4.17 (2H, td, J_{2′,1′}=4.8 Hz, H-2′), 4.12 (1H, t, J_{3,4}=2.7 Hz, H-3), 3.99 (2H, dq, J_{5,4}=6.1 Hz, J_{5a,5b}=12.6 Hz, H-5), 3.82 (1H, dd, J_{1′,2′}=4.6 Hz, H-1′a), 3.78(1H, dd, J_{1,2}=5.0 Hz, H-1a) 3.62 (1H, dd, J_{1,2}=5.0 Hz, H-1b), 3.60(1H, m, H-4), 3.51 (td, 1H, J_{1′,2′}=4.5 Hz, J_{1′a, 1′b=}13.8 Hz, H-1′b). ¹³C-NMR (D₂O, 125 MHz): δ 75.8 (C-3), 75.6(C-4), 73.8(C-2), 60.2 (d, ²J_{C-2, P}=4.7 Hz, C-2′), 59.4(C-1), 58.3(C-5), 57.01 (d, ³J_{C-1′, P}=3.9 Hz, C-1′). MALDI-TOF-MS: m/e 258.2 (M⁺+H). Anal. Calcd for C₇H₁₆NO₇P: C, 32.69; H, 6.27. Found.

### Hydroxypropyl-2,3,5-tri-O-benzyl-1,4-anhydro-4-thio-D-arabinitol (42)

The thioarabinitol (41) (4.8 mmol, 2.0 g) and 3-bromo-1-propanol (4.8 mmol, 0.43 mL) were dissolved in 1,1,1,3,3,3-hexafluoroisopropanol (HFIP) (2 mL) and the mixture was stirred in a sealed tube in an oil bath (92° C.) for 26 h (prolonged heating of the reaction mixture resulted in the formation of side products). As the resulting compound with a bromide counterion was moderately stable on silica gel, the bromide counterion was exchanged by dilution of the reaction mixture with dichloromethane, the addition of silver triflate (4.8 mmol, 1.2 g), and stirred for 2 h at ambient temperature. The solvents were removed by evaporation and the resulting residue was purified by column chromatography to give (42) as a colorless oil (1.82 g, 60%). [α]_{D}²³ −6.0° (c=1.0, MeOH). ¹H-NMR (CDCl₃, 500 MHz): δ 7.36-7.169 (15H, m, 3×Ph), 4.60-4.47 (6H, m, 2×CH₂Ph, HaCHPh, H-2), 4.40 (1H, d, J_{A,B}=11.5 Hz, HbCHPh) 5.2 Hz, H-2), 4.22-4.18(2H, m, H-2, H-1a), 3.97-3.90(2H, ddd, J_{5,4}=5.0 Hz, J_{5a,5b}=10 Hz 5-H), 3.77(1H, dd, J_{3′,2′}=6.0 Hz, H-3a′), 3.72-3.65(3H, m, H-3′b, H-4, H-1′a), 3.622(1H, dd, J_{1,2}=3.5 Hz, J_{1a,1b}=13.0 Hz, 1-Hb) 3.48(1H, td, J_{1′2′}=6.5 Hz, J_{1′a,1′b}=12.5 Hz, H-1′b), 2.12-1.99(2H, m, H-2′). ¹³C-NMR (CDCl₃, 125 MHz): δ 136.94, 136.24, 136.11(3×Cᵢₚₛₒ), 129.0-128.1(3×Ph), 128.3-119.6(CF₃ triflate), 83.0(C-3), 82.5(C-2), 73.9(CH₂Ph), 72.6(CH₂Ph), 72.1(CH₂Ph), 67.1(C-3′), 66.5(C-5), 59.5(C-4), 47.5 (C-1), 44.3(C-1′), 28.7(C-2′). MALDI-TOF-MS: m/e 479.03 (M⁺−OTf). Anal. Calcd for C₃₀H₃₅F₃O₇S₂ C, 57.31; H, 5.61. Found: C, 57.23; H, 5.59.

### 3′-Phosphorylpropyl-2,3,5-tri-O-benzyl-1,4-anhydro-4-thio-D-arabinitol (43)

Triphenylphosphine (2.4 mmol, 1.5 g), and diisopropyl azodicarboxylate (DIAD) (5.2 mmol, 0.46 mL) were stirred in anhydrous THF (5 mL) at 0° C. for 4 min. Dibenzyl phosphate (5.2 mmol, 1.46 g) was then added. After stirring the reaction mixture for an additional 4 min at 0° C., a solution of hydroxypropyl-2,3,5-tri-O-benzyl-1,4-dideoxy-1,4-thio-D-arabinitol (42) in THF (5 mL) was added dropwise at 0° C. under N₂, and the reaction mixture was stirred for 3 h at room temperature. After the completion of the reaction, THF was removed in vacuo and the residue was purified by column chromatography to give compound (43) as a colorless oil (1.7 g, 81%). [α]_{D}²³ +3.0° (c=1.0, MeOH). ¹H-NMR (CDCl₃, 500 MHz): δ 7.36-7.169 (15H, m, 3×Ph), 4.94-4.89 (4H, m, 2×POCH₂Ph), 4.60-4.47 (6H, m, 2×CH₂Ph, 1×H_{A}CHPh, H-2), 4.31 (1H, d, J_{A,B}=12.0 Hz, H_{b}CHPh), 4.18(1H, d, J_{1a,1b}=16.0 Hz, H-1a), 4.06 (1H, bs, H-3), 4.03-3.98(1H, m, H-3′a), 3.95(3H, m, H3′b, H-5a, H-4), 3.65-3.50(2H, m, H-5b, H-1′a), 3.48-3.3(2H, m, H-1′b, H-1b), 2.12-1.99(2H, m, H-2′). ¹³C-NMR (CDCl₃, 125 MHz): δ 136.94, 136.24, 136.11(3×Cᵢₚₛₒ), 129.0-128.1(3×Ph), 128.3-119.6(CF₃ triflate), 83.0(C-3), 82.5(C-2), 73.9(CH₂Ph), 72.6(CH₂Ph), 72.1(CH₂Ph), 69.93(d, J_{C,P}=5.75 Hz, POCH₂Ph) 66.9(C-4), 66.5(C-5), 65.07(d, ²J_{C,P}=5.75 Hz, C-3′), 47.5(C-1) 42.3(C-1′), 26.9(d, ³J_{C,P}=6.12 Hz, C-2′). MALDI-TOF-MS: m/e 739.08 (M⁺−OTf). Anal. Calcd for C₄₄H₄₈F₃O₁₀PS₂ C, 59.45; H, 5.44. Found: C, 59.11; H, 5.43.

### 3′-Phosphorylpropyl-1,4-anhydro-4-thio-D-arabinitol (37)

A solution of (43) (1.1 mmol, 1 g) in methanol (5 mL) containing 10% palladium on carbon (0.5 g) was stirred under 80 psi of hydrogen at room temperature for 16 h. The mixture was filtered, and the solvent was removed in vacuo to give the thio-alditol phosphate derivative as viscous oil. The compound was purified further by column chromatography (MeOH:H₂O, 8:1) to give (37) (0.25 g, 65%). [α]_{D}²³ −2.0° (c=1.1, MeOH). ¹H-NMR (D₂O, 500 MHz): δ 4.70 (1H, ddd, J_{2,3}=3.5 Hz, J_{2,1b}=7.0 Hz, H-2), 4.39 (1H, dd, J_{3,4}=3.0 Hz, H-3), 4.09 (1H, dd, J_{5,4}=4.5 Hz, H-5a), 4.03 (2H, ddd, J_{3′,2′}=6.0 Hz, H-3′), 3.97-3.94 (1H, m, H-4), 3.87 (1H, dd, J_{5,4}=4.4 Hz, H-5b), 3.89-3.76(2H, m, H-1), 3.67-3.56(2H, m, H-3′), 2.25-2.16(2H, m, H-2′). ¹³C-NMR (D₂O, 125 MHz): δ 119.7(q, J_{CF}=OCF₃), 77.7(C-3), 77.0(C-2), 69.8(C-4), 63.5(d, C-3′²J_{C-3′, P}=5.0 Hz), 59.3(C-5), 46.1(C-1), 42.3(C-1′), 26.4(d, C-2′, ³J_{C-2′, P}=4.5 Hz). MALDI-TOF-MS: m/e 289.2 (M⁺−OTf). Anal. Calcd for C₉H₁₈F₃O₁₀PS₂ C, 24.66; H, 4.14. Found: C, 24.32; H, 3.98.

5.3 S-Alkylated Analogues

General methods: Optical rotations were measured at 23° C. and reported in deg dm⁻¹ g⁻¹ cm³. ¹H and ¹³C NMR spectra were recorded at frequencies of 500 and 125 MHz, respectively. All assignments were confirmed with the aid of two-dimensional ¹H, ¹H (gCOSY) and ¹H, ¹³C (gHMQC) experiments using standard Varian pulse programs. Processing of the spectra was performed with MestRec software. 1D-NOESY experiments were recorded at 295K on a 500 MHz spectrometer. For each 1D-NOESY spectrum, 512 or 256 scans were acquired with a Q3 Gaussian Cascade pulse. A mixing time of 500 ms or 800 ms was used in the 1D-NOESY experiments. Matrix-assisted laser desorption ionization time-of-flight (MALDI-TOF) mass spectra were obtained on a PerSeptive Biosystems Voyager-DE spectrometer, using 2, 5-dihydroxybenzoic acid as a matrix. The high-resolution mass spectrum was recorded in positive-mode with turbo-ion spray ionization on a Hybrid Quadrupole-TOF LC/MS/MS mass spectrometer. Analytical thin-layer chromatography (TLC) was performed on aluminum plates precoated with silica gel 60F-254 as the adsorbent. The developed plates were air-dried, exposed to UV light and/or sprayed with a solution containing 1% Ce (SO₄)₂ and 1.5% molybdic acid in 10% aqueous H₂SO₄, and heated. Column chromatography was performed with silica gel 60 (230-400 mesh).

Enzyme activity assay: IC₅₀ values for inhibition of MGA with compounds 44-52 were determined using the p-nitrophenyl-D-glucopyranoside (pNP-glucose, Sigma) assay to follow the production of p-nitrophenol upon addition of enzyme (500 nM).

The assays were carried out in 96-well microtiter plates containing 100 mM MES buffer pH 6.5, inhibitor and pNP-glucose as substrate, with a final volume of 50 μL. Reactions were incubated at 37° C. for 35 min and terminated by addition of 50 μL of 0.5 M sodium carbonate. The absorbance of the reaction product was measured at 405 nm in a microtiter plate reader. All reactions were performed in triplicate and absorbance measurements were averaged to give a final result.

### 1-Ethoxy-6-iodohexane (60)

A solution of 6-bromohexan-1-ol (59) (1 g, 5.5 mmol) was dissolved in EtOH (20 mL) and NaOEt (3.76 g, 55.2 mmol) was added. The reaction mixture was refluxed with vigorous stirring for 12 h. The reaction was monitored by TLC analysis of aliquots (30% EtOAc/Hexanes). When the limiting reagents had been essentially consumed, the mixture was cooled and the solvent was evaporated to give a syrupy residue that was passed through a pad of silica to give the corresponding ethoxy alcohol (800 mg, crude) that was subsequently treated with MsCl (0.895 mL, 11.5 mmol) and Et₃N (1.98 mL, 14.3 mmol) in CH₂Cl₂ (30 mL) at 0° C. After the consumption of starting material, as indicated by TLC after 7 h, the reaction mixture was diluted with CH₂Cl₂ (15 mL) and washed with water (2×10 mL). The organic layer was dried over anhydrous Na₂SO₄ and concentrated to give the corresponding mesylate. The crude mesylate (800 mg) was dissolved in dry acetone (15 mL), and freshly fused NaI (1.6 g, 10.7 mmol) was added. The reaction mixture was heated at 55° C. for 6 h, cooled, and the solvents were removed by evaporation at atmospheric pressure (at 25° C.); the residue was then purified by chromatography to give the title compound as a colorless oil (850 mg, 60%); ¹H NMR (CDCl₃): δ 3.47 (q, 2H, J=7 Hz, —OCH₂CH₃), 3.41 (t, 2H, J_{1,2}=7 Hz, H-1), 3.19 (t, 2H, J_{6,5}=7 Hz, H-6), 1.86-1.80 (m, 2H, H-5), 1.61-1.55 (m, 2H, H-2), 1.45-1.34 (m, 4H, H-3 and H-4), 1.20 (t, 3H, J=7 Hz, —OCH₂CH₃); ¹³C NMR (CDCl₃): δ 70.4 (C-1), 66.1 (—OCH₂CH₃), 33.4 (C-5), 30.3, 29.6 (2C, C-2 and C-4), 25.2 (C-3), 15.2 (—OCH₂CH₃), 7.1 (C-6); Anal. Calcd for C₈H₁₇IO: C, 37.52; H, 6.69. Found: C, 37.75; H, 6.89.

### 1-Iodo-9-methoxynonane (62)

A solution of 9-bromononan-1-ol (61) (1.1 g, 4.9 mmol) was dissolved in MeOH (20 mL) and NaOMe (2.66 g, 49.3 mmol) was added. The reaction mixture was refluxed with vigorous stirring for 12 h. The reaction was monitored by TLC analysis of aliquots (30% EtOAc/Hexanes). When the limiting reagents had been essentially consumed, the mixture was cooled and the solvent was evaporated to give a syrupy residue that was passed through a pad of silica to give the corresponding methoxy alcohol (830 mg, crude) that was subsequently treated with MsCl (0.775 mL, 10.0 mmol) and Et₃N (1.72 mL, 12.4 mmol) in CH₂Cl₂ (30 mL) at 0° C. After the consumption of starting material, as indicated by TLC after 7 h, the reaction mixture was diluted with CH₂Cl₂ (15 mL) and washed with water (2×10 mL). The organic layer was dried over anhydrous Na₂SO₄ and concentrated to give the corresponding mesylate. The crude mesylate (890 mg) was dissolved in dry acetone (15 mL), and freshly fused NaI (3.2 g, 21.2 mmol) was added. The reaction mixture was heated at 55° C. for 5 h, cooled, and the solvents were removed by evaporation at atmospheric pressure (at 25° C.); the residue was then purified by chromatography to give the title compound as a colorless oil (820 mg, 59%). This material was identical in all respects to that reported previously.^{64 1}H NMR (CDCl₃): δ 3.36 (t, 2H, J_{9,8}=7 Hz, H-9), 3.33 (s, 3H, —OCH₃), 3.18 (t, 2H, J_{1,2}=7 Hz, H-1), 1.84-1.78 (m, 2H, H-2), 1.59-1.53 (m, 2H, H-8), 1.41-1.25 (m, 10H, H-3-H-7); ¹³C NMR (CDCl₃): δ 72.7 (C-9), 58.3 (—OCH₃), 33.4 (C-2), 30.3, 29.4 (2C, C-3 and C-8), 29.2, 29.1, 28.3 (3C, C-4, C-5 and C-6), 26.0 (C-7), 7.0 (C-1).

### 2,3,5-Tri-O-benzyl-1,4-dideoxy-1,4-[(1-tetradecyl)-(R)-episulfoniumylidene]-D-arabinitol triflate (65)

A mixture of the thioarabinitol 63 (500 mg, 1.19 mmol) and the bromide 54 (0.357 mL, 1.31 mmol) in 1,1,1,3,3,3-hexafluoro-2-propanol (HFIP, 1 mL) was stirred and heated in a sealed tube at 90° C. for 24 h. The progress of the reaction was monitored by TLC analysis of aliquots (CHCl₃/MeOH, 10:1) that showed a 30% conversion of product. Further heating did not improve the conversion rate. Hence, the mixture was cooled, and concentrated to give a syrupy residue. Purification by column chromatography (gradient of CHCl₃/MeOH, 10:1) gave the purified sulfonium-ion 64 (235 mg, 47%, based on the isolation of 40% unreacted starting material 63); treatment with silver triflate (86.6 mg, 0.337 mmol) in CH₂Cl₂ at ambient temperature for 5 h gave the sulfonium ion 65 as a colorless syrup (240 mg, 93%): [α]_{D} +4.44 (c 0.2, MeOH); ¹H NMR (CDCl₃): δ 7.36-7.32 (m, 9H, Ar), 7.25-7.18 (m, 6H, Ar), 4.62 and 4.52 (2d, each 1H, J_{a,b}=11.9 Hz, CH₂Ph), 4.61 and 4.43 (2d, each 1H, J_{a,b}=11.8 Hz, CH₂Ph), 4.53 and 4.45 (2d, each 1H, J_{a,b}=11.7 Hz, CH₂Ph), 4.53 (br s, 1H, H-2), 4.41 (d, 1H, J_{1a,1b}=12.9 Hz, H-1a), 4.17 (br s, 1H, H-3), 3.81-3.76 (m, 2H, H-4 and H-5a), 3.74 (ddd, 1H, J_{1′a,1′b}=12.7 Hz, J_{1′a,2′a}=J_{1′a,2′b}=8.1 Hz, H-1′a), 3.64 (dd, 1H, J_{5b,4}=J_{5b,5a}=12.1 Hz, H-5b ), 3.64 (dd, 1H, J_{1b,1a}=12.9 Hz, J_{1b,2}=2.1 Hz, H-1b), 3.30-3.24 (m, 1H, H-1′b), 1.78-1.72 (m, 2H, H-2′), 1.50-1.20 (m, 22H, H-3′-H-13′), 0.88 (dd, 3H, J_{14′,13′a}=J_{14′,13′b}=7 Hz, H-14′); ¹³C NMR (CDCl₃): δ 136.5, 135.9, 135.7 (3Cᵢₚₛₒ), 128.9-127.9 (15C_{Ar}), 120.7 (q, IC, J_{C,F}=319 Hz, OTf), 83.1 (C-3), 81.7 (C-2), 73.8, 72.7, 71.9 (3 CH₂Ph), 66.9 (C-5), 66.3 (C-4), 47.1 (C-1), 45.7 (C-1′), 31.9-22.7 (11C, C-3′-C-13′), 25.7 (C-2′), 14.1 (C-14′); MALDI-TOF MS: m/z 617.54 [M−OTf]⁺. Anal. Calcd for C₄₁H₅₇F₃O₆S₂: C, 64.20; H, 7.49. Found: C, 63.91; H, 7.58.

### General Procedure for the Preparation of Sulfonium-Ions 66-73

To a mixture of the thioarabinitol 63 and the alkyl halide (53-58, 60 or 62) in dry CH₃CN was added AgBF₄ (1 equiv. with respect to alkyl halide), and the stirred reaction mixture was heated at 65° C. for 17 h. The progress of the reaction was monitored by TLC analysis of aliquots (CHCl₃/MeOH, 10:1). When the limiting reagent had been essentially consumed, the mixture was cooled, and concentrated to give a syrupy residue. Purification by column chromatography (gradient of CHCl₃/MeOH, 10:1) gave the purified sulfonium-ions 66-73.

### 2,3,5-Tri-O-benzyl-1,4-dideoxy-1,4-[(1-tetradecyl)-(R)-episulfoniumylidene]-D-arabinitol tetrafluoroborate (66)

The reaction of thioarabinitol 63 (300 mg, 0.71 mmol) with the bromide 54 (0.195 mL, 0.79 mmol) and AgBF₄ (154 mg, 0.79 mmol) in dry CH₃CN (8 mL) gave compound 66 as a colorless syrup (370 mg, 74%): [α]_{D} +8.11 (c 0.4, MeOH); ¹H NMR (CDCl₃): δ 7.37-7.32 (m, 9H, Ar), 7.25-7.18 (m, 6H, Ar), 4.62 and 4.50 (2d, each 1H, J_{a,b}=11.9 Hz, CH₂Ph), 4.61 and 4.43 (2d, each 1H, J_{a,b}=11.8 Hz, CH₂Ph), 4.54 and 4.48 (2d, each 1H, J_{a,b}=11.7 Hz, CH₂Ph), 4.54 (br s, 1H, H-2), 4.37 (d, 1H, J_{1a,1b}=13.2 Hz, H-1a), 4.15 (br s, 1H, H-3), 3.78-3.75 (m, 2H, H-4 and H-5a), 3.68 (ddd, 1H, J_{1′a,1′b}=13.1 Hz, J_{1′a,2′a}=J_{1′a,2′b}=8.2 Hz, H-1′a), 3.64 (dd, 1H, J_{5b,4}=J_{5b,5a}=11.9 Hz, H-5b ), 3.64 (dd, 1H, J_{1b,1a}=13.2 Hz, J_{1b,2}=3.1 Hz, H-1b), 3.28-3.22 (m, 1H, H-1′b), 1.78-1.72 (m, 2H, H-2′), 1.50-1.20 (m, 22H, H-3′-H-13′), 0.88 (dd, 3H, J_{14′,13′a}=J_{14′,13′b}=6.8 Hz, H-14′); ¹³C NMR (CDCl₃): δ 136.6, 136, 135.8 (3Cᵢₚₛₒ), 128.8-127.8 (15C_{Ar}), 82.8 (C-3), 81.8 (C-2), 73.7, 72.6, 71.9 (3 CH₂Ph), 66.9 (C-5), 66.6 (C-4), 46.8 (C-1), 45.4 (C-1′), 31.9-22.7 (11C, C-3′-C-13′), 25.7 (C-2′), 14.1 (C-14′); MALDI-TOF MS: m/z 617.86 [M−BF₄]⁺. Anal. Calcd for C₄₀H₅₇BF₄O₃S: C, 68.17; H, 8.15. Found: C, 68.14; H, 8.29.

### 2,3,5-Tri-O-benzyl-1,4-dideoxy-1,4-[(1-octyl)-(R)-episulfoniumylidene]-D-arabinitol tetrafluoroborate (67)

The reaction of thioarabinitol 63 (500 mg, 1.19 mmol) with the bromide 53 (253 mg, 1.31 mmol) and AgBF₄ (255 mg, 1.31 mmol) in dry CH₃CN (12 mL) gave compound 67 as a colorless syrup (590 mg, 79%): [α]_{D} +8.16 (c 0.5, MeOH); ¹H NMR (CDCl₃): δ 7.36-7.31 (m, 9H, Ar), 7.25-7.18 (m, 6H, Ar), 4.64 and 4.52 (2d, each 1H, J_{a,b}=11.9 Hz, CH₂Ph), 4.60 and 4.45 (2d, each 1H, J_{a,b}=11.8 Hz, CH₂Ph), 4.53 and 4.48 (2d, each 1H, J_{a,b}=11.7 Hz, CH₂Ph), 4.58 (br s, 1H, H-2), 4.32 (d, 1H, J_{1a,1b}=13.2 Hz, H-1a), 4.18 (br s, 1H, H-3), 3.82-3.77 (m, 2H, H-4 and H-5a), 3.65 (dd, 1H, J_{5b,4}=J_{5b,5a}=11.4 Hz, H-5b ), 3.64 (dd, 1H, J_{1b,1a}=13.2 Hz, J_{1b,2}=3.7 Hz, H-1b), 3.64 (ddd, 1H, J_{1′a,1′b}=13.1 Hz, J_{1′a,2′a}=J_{1′a,2′b}=7.5 Hz, H-1′a), 3.28-3.21 (m, 1H, H-1′b), 1.78-1.71 (m, 2H, H-2′), 1.50-1.19 (m, 10H, H-3′-H-7′), 0.87 (dd, 3H, J_{8′,7′a}=J_{8′,7′b}=7 Hz, H-8′); ¹³C NMR (CDCl₃): δ 136.6, 136.1, 135.9 (3Cᵢₚₛₒ), 128.8-127.9 (15C_{Ar}), 82.8 (C-3), 81.9 (C-2), 73.7, 72.6, 71.9 (3 CH₂Ph), 66.9 (C-5), 66.7 (C-4), 46.8 (C-1), 45.4 (C-1′), 31.6-22.5 (5C, C-3′-C-7′), 25.6 (C-2′), 14.0 (C-8′); MALDI-TOF MS: m/z 532.95 [M−BF₄]⁺. Anal. Calcd for C₃₄H₄₅BF₄O₃S: C, 65.80; H, 7.31. Found: C, 65.92; H, 7.26.

### 2,3,5-Tri-O-benzyl-1,4-dideoxy-1,4-[(1-octadecyl)-(R)-episulfoniumylidene]-D-arabinitol tetrafluoroborate (68)

The reaction of thioarabinitol 63 (500 mg, 1.19 mmol) with the bromide 55 (436 mg, 1.31 mmol) and AgBF₄ (255 mg, 1.31 mmol) in dry CH₃CN (12 mL) gave compound 68 as a colorless syrup (465 mg, 73%, based on the isolation of 30% unreacted starting material 63): [α]_{D} +6.67 (c 0.3, MeOH); ¹H NMR (CDCl₃): δ 7.37-7.29 (m, 9H, Ar), 7.25-7.18 (m, 6H, Ar), 4.63 and 4.52 (2d, each 1H, J_{a,b}=11.9 Hz, CH₂Ph), 4.60 and 4.44 (2d, each 1H, J_{a,b}=11.8 Hz, CH₂Ph), 4.58 (br s, 1H, H-2), 4.53 and 4.48 (2d, each 1H, J_{a,b}=11.7 Hz, CH₂Ph), 4.32 (d, 1H, J_{1a,1b}=13.2 Hz, H-1a), 4.18 (br s, 1H, H-3), 3.81-3.78 (m, 2H, H-4 and H-5a), 3.67-3.62 (m, 1H, H-1′a), 3.64 (dd, 1H, J_{5b,5a}=J_{5b,4}=11.0 Hz, H-5b), 3.64 (dd, 1H, J_{1b,1a}=13.2 Hz, J_{1b,2}=3.6 Hz, H-1b), 3.28-3.20 (m, 1H, H-1′b), 1.77-1.71 (m, 2H, H-2′), 1.50-1.20 (m, 30H, H-3′-H-17′), 0.88 (dd, 3H, J_{18′,17′a}=J_{18′,17′b}=6.8 Hz, H-18′); ¹³C NMR (CD₃Cl₃): δ 136.6, 136.1, 135.9 (3Cᵢₚₛₒ), 128.8-127.8 (15C_{Ar}), 82.8 (C-3), 82.0 (C-2), 73.6, 72.6, 71.8 (3 CH₂Ph), 66.9 (C-5), 66.7 (C-4), 46.8 (C-1), 45.3 (C-1′), 31.9-22.6 (15C, C-3′-C-17′), 25.6 (C-2′), 14.1 (C-18′); MALDI-TOF MS: m/z 673.57 [M−BF₄]⁺. Anal. Calcd for C₄₄H₆₅BF₄O₃S: C, 69.46; H, 8.61. Found: C, 69.64; H, 8.53.

### 2,3,5-Tri-O-benzyl-1,4-dideoxy-1,4-[[1-(3-methyl)-butyl]-(R)-episulfoniumylidene]-D-arabinitol tetrafluoroborate (69)

The reaction of thioarabinitol 63 (580 mg, 1.38 mmol) with the bromide 56 (0.182 mL, 1.52 mmol) and AgBF₄ (296 mg, 1.52 mmol) in dry CH₃CN (14 mL) gave compound 69 as a colorless syrup (518 mg, 65%): [α]_{D} +6.67 (c 0.5, MeOH); ¹H NMR (CDCl₃): δ 7.37-7.29 (m, 9H, Ar), 7.25-7.18 (m, 6H, Ar), 4.62 and 4.50 (2d, each 1H, J_{a,b}=11.9 Hz, CH₂Ph), 4.61 and 4.44 (2d, each 1H, J_{a,b}=11.8 Hz, CH₂Ph), 4.54 and 4.49 (2d, each 1H, J_{a,b}=11.7 Hz, CH₂Ph), 4.54 (br s, 1H, H-2), 4.38 (d, 1H, J_{1a,1b}=13.1 Hz, H-1a), 4.15 (br s, 1H, H-3), 3.78 (dd, 1H, J_{4,5a}=5 Hz, J_{4,5b}=11.5 Hz, H-4), 3.78 (dd, 1H, J_{5a,4}=5 Hz, J_{5a,5b}=11.5 Hz, H-5a), 3.70 (ddd, 1H, J_{1′a,1′b}=12.5 Hz, J_{1′a,2′a}=10.5 Hz, J_{1′a,2′b}=6.5 Hz, H-1′a), 3.64 (dd, 1H, J_{5b,4}=J_{5b,5a}=11.5 Hz, H-5b), 3.64 (dd, 1H, J_{1b,1a}=13.2 Hz, J_{1b,2}=3.5 Hz, H-1b), 3.24 (ddd, 1H, J_{1′b,1′′a}=12.5 Hz, J_{1′b,2′a}=9.5 Hz, J_{1′b,2′b}=5 Hz, H-1′b), 1.75-1.55 (m, 3H, H-2′ and H-3′), 0.91 and 0.87 (2d, each 3H, J_{4′,3′}=J_{5′,3′}=6.5 Hz, H-4′ and H-5′); ¹³C NMR (CDCl₃): δ 136.6, 136.1, 135.9 (3Cᵢₚₛₒ), 128.8-127.9 (15C_{Ar}), 82.8 (C-3), 82.0 (C-2), 73.6, 72.6, 71.8 (3 CH₂Ph), 66.9 (C-5), 66.6 (C-4), 46.8 (C-1), 43.5 (C-1′), 33.9 (C-2′), 27.1 (C-3′), 21.9, 21.5 (2C, C-4′ and C-5′); MALDI-TOF MS: m/z 491.18 [M−BF₄]⁺. Anal. Calcd for C₃₁H₃₉BF₄O₃S: C, 64.36; H, 6.79. Found: C, 64.39; H, 6.99.

### 2,3,5-Tri-O-benzyl-1,4-dideoxy-1,4-[(1-butyl)-(R)-episulfoniumylidene]-D-arabinitol tetrafluoroborate (70)

The reaction of thioarabinitol 63 (500 mg, 1.19 mmol) with the iodide 57 (0.162 mL, 1.43 mmol) and AgBF₄ (276 mg, 1.43 mmol) in dry CH₃CN (14 mL) gave compound 70 as a colorless syrup (600 mg, 90%): [α]_{D} +4.12 (c 0.5, MeOH); ¹H NMR (CDCl₃): δ 7.35-7.30 (m, 9H, Ar), 7.25-7.18 (m, 6H, Ar), 4.64 and 4.55 (2d, each 1H, J_{a,b}=11.9 Hz, CH₂Ph), 4.58 and 4.45 (2d, each 1H, J_{a,b}=11.8 Hz, CH₂Ph), 4.52 and 4.49 (2d, each 1H, J_{a,b}=11.8 Hz, CH₂Ph), 4.57 (d, 1H, J_{2,1b}=2.6 Hz, H-2), 4.27 (d, 1H, J_{1a,1b}=13.1 Hz, H-1a), 4.24 (br s, 1H, H-3), 3.83 (dd, 1H, J_{4,5a}=5 Hz, J_{4,5b}=11.3 Hz, H-4), 3.83 (dd, 1H, J_{5a,4}=5 Hz, J_{5a,5b}=11.3 Hz, H-5a), 3.65 (dd, 1H, J_{5b,4}=J_{5b,5a}=11.3 Hz, H-5b ), 3.62 (dd, 1H, J_{1b,1a}=13.1 Hz, J_{1b,2}=2.7 Hz, H-1b), 3.62 (ddd, 1H, J_{1′a,1′b}=13.0 Hz, J_{1′a,2′a}=J_{1′a,2′b}=7.5 Hz, H-1′a), 3.27-3.21 (m, 1H, H-1′b), 1.78-1.70 (m, 2H, H-2′), 1.49-1.39 (m, 2H, H-3′), 0.85 (dd, 3H, J_{4′,3′a}=J_{4′,3′b}=7.5 Hz, H-4′); ¹³C NMR (CDCl₃): δ 136.7, 136.2, 135.9 (3Cᵢₚₛₒ), 128.7-127.8 (15C_{Ar}), 82.7 (C-3), 82.1 (C-2), 73.6, 72.5, 71.8 (3 CH₂Ph), 66.8 (C-5), 66.6 (C-4), 46.7 (C-1), 45.1 (C-1′), 27.4 (C-2′), 21.1 (C-3′), 13.1 (C-4′); MALDI-TOF MS: m/z 477.33 [M−BF₄]⁺. Anal. Calcd for C₃₀H₃₇BF₄O₃S: C, 63.83; H, 6.61. Found: C, 63.50; H, 6.57.

### 2,3,5-Tri-O-benzyl-1,4-dideoxy-1,4-[(1-hexyl)-(R)-episulfoniumylidenel-D-arabinitol tetrafluoroborate (71)

The reaction of thioarabinitol 63 (610 mg, 1.45 mmol) with the iodide 58 (0.211 mL, 1.43 mmol) and AgBF₄ (276 mg, 1.43 mmol) in dry CH₃CN (14 mL) gave compound 71 as a colorless syrup (810 mg, 95%): [α]_{D} +4.00 (c 0.8, MeOH); ¹H NMR (CD₃OD): δ 7.37-7.24 (m, 15H, Ar), 4.65 and 4.62 (2d, each 1H, J_{a,b}=11.7 Hz, CH₂Ph), 4.64 (br s, 1H, H-2), 4.56 and 4.47 (2d, each 1H, J_{a,b}=11.6 Hz, CH₂Ph), 4.54 and 4.50 (2d, each 1H, J_{a,b}=11.7 Hz, CH₂Ph), 4.39 (br s, 1H, H-3), 4.13 (dd, 1H, J_{4,5a}=5 Hz, J_{4,5b}=11.0 Hz, H-4), 4.04 (d, 1H, J_{1a,1b}=12.5 Hz, H-1a), 3.88 (dd, 1H, J_{5a,4}=5 Hz, J_{5a,5b}=10.5 Hz, H-5a), 3.67 (dd, 1H, J_{1b,1a}=12.5 Hz, J_{1b,2}=3 Hz, H-1b), 3.64 (dd, 1H, J_{5b,5a}=10.5 Hz, J_{5b,4}=11.0 Hz, H-5b), 3.41 (ddd, 1H, J_{1′a,1′b}=12.5 Hz, J_{1′a,2′a}=J_{1′a,2′b}=8.0 Hz, H-1′a), 3.31(ddd, 1H, J_{1′b,1′a}=12.5 Hz, J_{1′b,2′a}=5.5 Hz, J_{1′b,2′b}=8.5 Hz, H-1′b), 1.82-1.68 (m, 2H, H-2′), 1.43-1.40 (m, 2H, H-3′), 1.21-1.18 (m, 4H, H-4′ and H-5′), 0.85 (dd, 3H, J_{6′,5′a}=J_{6′,5′b}=7.0 Hz, H-6′); ¹³C NMR (CD₃OD): δ 138.5, 138.3, 138.0 (3Cᵢₚₛₒ), 129.8-129.2 (15C_{Ar}), 84.3 (C-3), 84.2 (C-2), 74.6, 73.6, 73.1 (3 CH₂Ph), 68.2 (C-5), 67.7 (C-4), 47.9 (C-1), 46.7 (C-1′), 32.2, 23.4 (C-4′ and C-5′), 28.8 (C-3′), 26.7 (C-2′), 14.3 (C-6′); MALDI-TOF MS: m/z 505.34 [M−BF₄]⁺. Anal. Calcd for C₃₂H₄₁BF₄O₃S: C, 64.86; H, 6.97. Found: C, 64.78; H, 7.10.

### 2,3,5-Tri-O-benzyl-1,4-dideoxy-1,4-[[1-(6-ethoxy)-hexyl]-(R)-episulfoniumylidene]-D-arabinitol tetrafluoroborate (72)

The reaction of thioarabinitol 63 (500 mg, 1.19 mmol) with the iodide 60 (363 mg, 1.42 mmol) and AgBF₄ (276 mg, 1.43 mmol) in dry CH₃CN (14 mL) gave compound 72 as a colorless syrup (680 mg, 90%): [α]_{D} +8.1 (c 0.6, MeOH); ¹H NMR (CDCl₃): δ 7.38-7.33 (m, 9H, Ar), 7.25-7.18 (m, 6H, Ar), 4.61 and 4.50 (2d, each 1H, J_{a,b}=11.9 Hz, CH₂Ph), 4.60 and 4.43 (2d, each 1H, J_{a,b}=11.8 Hz, CH₂Ph), 4.54 and 4.48 (2d, each 1H, J_{a,b}=11.7 Hz, CH₂Ph), 4.54 (br s, 1H, H-2), 4.37 (d, 1H, J_{1a,1b}=13.2 Hz, H-1a), 4.14 (br s, 1H, H-3), 3.79-3.74 (m, 2H, H-4 and H-5a), 3.70 (ddd, 1H, J_{1′a,1′b}=12.5 Hz, J_{1′a,2′a}=J_{1′a,2′b}=8.0 Hz, H-1′a), 3.63 (dd, 1H, J_{5b,4}=J_{5b,5a}=12.5 Hz, H-5b ), 3.63 (dd, 1H, J_{1b,1a}=13.2 Hz, J_{1b,2}=3.0 Hz, H-1b), 3.45 (q, 2H, J=7 Hz, —OCH₂CH₃), 3.36 (dd, 2H, J_{6′,5′a}=J_{6′,5′b}=6.5 Hz, H-6′), 3.29-3.23 (m, 1H, H-1′b), 1.80-1.74 (m, 2H, H-2′), 1.53-1.40 (m, 4H, H-3′ and H-5′), 1.39-1.27 (m, 2H, H-4′), 1.18 (t, 3H, J=7 Hz, —OCH₂CH₃); ¹³C NMR (CDCl₃): δ 136.6, 136.1, 135.9 (3Cᵢₚₛₒ), 128.8-127.9 (15C_{Ar}), 82.7 (C-3), 82.0 (C-2), 73.6, 72.6, 71.8 (3 CH₂Ph), 70.1 (C-6′), 66.8 (C-5), 66.7 (C-4), 66.0 (—OCH₂CH₃), 46.8 (C-1), 45.2 (C-1′), 29.2 (C-5′), 27.7 (C-3′), 25.5 (C-2′), 25.4 (C-4′), 15.2 (—OCH₂CH₃); MALDI-TOF MS: m/z 549.49 [M−BF₄]⁺. Anal. Calcd for C₃₄H₄₅BF₄O₄S: C, 64.15; H, 7.13. Found: C, 64.36; H, 7.33.

### 2,3,5-Tri-O-benzyl-1,4-dideoxy-1,4-[[1-(9-methoxy)-nonyl]-(R)-episulfoniumylidene]-D-arabinitol tetrafluoroborate (73)

The reaction of thioarabinitol 63 (740 mg, 1.76 mmol) with the iodide 62 (600 mg, 2.11 mmol) and AgBF₄ (411 mg, 2.11 mmol) in dry CH₃CN (12 mL) gave compound 73 as a colorless syrup (1.05 g, 89%): [α]_{D} +7.30 (c 0.3, MeOH); ¹H NMR (CDCl₃): δ 7.37-7.33 (m, 9H, Ar), 7.25-7.18 (m, 6H, Ar), 4.61 and 4.50 (2d, each 1H, J_{a,b}=12.0 Hz, CH₂Ph), 4.60 and 4.43 (2d, each 1H, J_{a,b}=11.8 Hz, CH₂Ph), 4.54 and 4.48 (2d, each 1H, J_{a,b}=11.5 Hz, CH₂Ph), 4.54 (br s, 1H, H-2), 4.37 (d, 1H, J_{1a,1b}=13.0 Hz, H-1a), 4.14 (br s, 1H, H-3), 3.80-3.75 (m, 2H, H-4 and H-5a), 3.68 (ddd, 1H, J_{1′a,1′b}=13.0 Hz, J_{1′a,2′a}=J_{1′a,2′b}=8.0 Hz, H-1′a), 3.64 (dd, 1H, J_{5b,4}=J_{5b,5a}=12.0 Hz, H-5b ), 3.64 (dd, 1H, J_{1b,1a}=13.0 Hz, J_{1b,2}=2.8 Hz, H-1b), 3.36 (dd, 2H, J_{9′,8′a}=J_{9′,8′b}=6.8 Hz, H-9′), 3.33 (s, 3H, —OCH₃), 3.28-3.23 (m, 1H, H-1′b), 1.78-1.72 (m, 2H, H-2′), 1.56-1.52 (m, 2H, H-8′), 1.49-1.37 (m, 2H, H-3′), 1.34-1.18 (m, 8H, H-4′, H-5′, H-6′ and H-7′); ¹³C NMR (CDCl₃): δ 136.6, 136.1, 135.9 (3Cᵢₚₛₒ), 128.8-127.9 (15C_{Ar}), 82.8 (C-3), 81.9 (C-2), 73.6, 72.6, 71.8 (3 CH₂Ph), 72.8 (C-9′), 66.8 (C-5), 66.7 (C-4), 58.5 (—OCH₃), 46.7 (C-1), 45.3 (C-1′), 29.5 (C-8′), 29.2, 29.1, 28.7, 26.0 (4C, C-4′-C-7′), 27.9 (C-3′), 25.6 (C-2′); MALDI-TOF MS: m/z 577.04 [M−BF₄]⁺. Anal. Calcd for C₃₆H₄₉BF₄O₄S: C, 65.06; H, 7.43. Found: C, 65.13; H, 7.53.

### 1,4-Dideoxy-1,4-[(1-tetradecyl)-(R)-episulfoniumylidene]-D-arabinitol triflate (44)

BCl₃ gas was bubbled vigorously through a solution of the sulfonium-ion 65 (200 mg, 0.26 mmol) in CH₂Cl₂ (5 mL) at −78° C. under N₂ atmosphere for 5 min. The mixture was stirred at −78° C. for 1.5 h and a stream of dry air was blown vigorously over the solution to remove excess BCl₃. The reaction was quenched with MeOH (3 mL), and the solvents were removed. The residue was co-evaporated with MeOH (2×4 mL) to give the final product 44, as a colorless, amorphous solid (120 mg, 93%): mp 69-71° C.; [α]_{D} +19.5 (c 0.2, MeOH); ¹H NMR (CD₃OD): δ 4.60 (br s, 1H, H-2), 4.31 (d, 1H, J_{3,2}=1.5 Hz, H-3), 4.03-4.0 (m, 1H, H-5a), 3.86-3.80 (m, 2H, H-4 and H-5b), 3.81 (d, 1H, J_{1a,1b}=12.5 Hz, H-1a), 3.70 (dd, 1H, J_{1b,1a}=12.5 Hz, J_{1b,2}=3 Hz, H-1b), 3.50 (ddd, 1H, J_{1′a,1′b}=12.5 Hz, J_{1′a,2′a}=J_{1′a,2′b}=8.0 Hz, H-1′a), 3.40 (ddd, 1H, J_{1′b,1′a}=12.5 Hz, J_{1′b,2′a}=8.5 Hz, J_{1′b,2′b}=5.5 Hz, H-1′b), 1.91-1.80 (m, 2H, H-2′), 1.55-1.42 (m, 2H, H-3′), 1.41-1.22 (m, 20H, H-4′-H-13′), 0.87 (dd, 3H, J_{14′,13′a}=J_{14′,13′b}=7.0 Hz, H-14′); ¹³C NMR (CD₃OD): δ 121.8 (q, 2C, J_{C,F}=316.3 Hz, OTt), 79.6 (C-3), 79.5 (C-2), 73.6 (C-4), 61.0 (C-5), 50.1 (C-1), 46.7 (C-1′), 33.1-23.8 (10C, C-4′-C-13′), 29.2 (C-3′), 26.8 (C-2′), 14.5 (C-14′); MALDI-TOF MS: m/z 347.25 [M−OTf]⁺. Anal. Calcd for C₂₀H₃₉F₃O₆S₂: C, 48.37; H, 7.92. Found: C, 47.97; H, 7.77.

### General Procedure for the Deprotection of the S-Alkylated Sulfonium-Ions (66-73)

BCl₃ gas was bubbled vigorously through a solution of the sulfonium-ion (66-73) in CH₂Cl₂ at −78° C. under N₂ atmosphere for 10 min. The mixture was stirred at −78° C. for 2 h and a stream of dry air was blown vigorously over the solution to remove excess BCl₃. The reaction was quenched with MeOH (3-4 mL), and the solvents were removed. The residue was co-evaporated with MeOH (2×4 ml) to give the deprotected products which were subsequently dissolved in MeOH and a freshly washed ion-exchange resin Amberlyst A-26 (chloride form) was added. The mixture was stirred at room temperature for 2 h and filtered. The filtrate was concentrated to give the final products 45-52.

### 1,4-Dideoxy-1,4-[(1-butyl)-(R)-episulfoniumylidene]-D-arabinitol chloride (45)

Treatment of the sulfonium-ion 70 (560 mg, 0.99 mmol) with BCl₃ in CH₂Cl₂ (12 mL), followed by treatment with Amberlyst A-26 resin (250 mg) gave the final product 45 as a colorless syrup (230 mg, 96%): [α]_{D} +21.05 (c 0.2, MeOH); ¹H NMR (CD₃OD): δ 4.62 (br s, 1H, H-2), 4.33 (br s, 1H, H-3), 4.10-4.0 (m, 1H, H-5a), 3.90-3.83 (m, 2H, H-4 and H-5b), 3.84 (d, 1H, J_{1a,1b}=12.5 Hz, H-1a), 3.71 (dd, 1H, J_{1b,1a}=12.5 Hz, J_{1b,2}=3.0 Hz, H-1b), 3.50 (ddd, 1H, J_{1′a,1′b}=12.5 Hz, J_{1′a,2′a}=J_{1′a,2′b}=8.0 Hz, H-1′a), 3.42 (ddd, 1H, J_{1′b,1′a}=12.5 Hz, J_{1′b,2′a}=8.5 Hz, J_{1′b,2′b}=6.2 Hz, H-1′b), 1.90-1.80 (m, 2H, H-2′), 1.60-1.48 (m, 2H, H-3′), 1.0 (dd, 3H, J_{4′,3′a}=J_{4′,3′b}=7.0 Hz, H-4′); ¹³C NMR (CD₃OD): δ 79.6 (C-3), 79.4 (C-2), 73.6 (C-4), 61.0 (C-5), 50.2 (C-1), 46.4 (C-1′), 28.7 (C-2′), 22.5 (C-3′), 13.7 (C-4′); MALDI-TOF MS: m/z 207.36 [M−Cl]⁺. Anal. Calcd for C₉H₁₉ClO₃S: C, 44.53; H, 7.89. Found: C, 44.28; H, 8.11.

### 1,4-Dideoxy-1,4-[(1-hexyl)-(R)-episulfoniumylidene]-D-arabinitol chloride (46)

Treatment of the sulfonium-ion 71 (425 mg, 0.72 mmol) with BCl₃ in CH₂Cl₂ (9 mL), followed by treatment with Amberlyst A-26 resin (200 mg) gave the final product 46 as a colorless syrup (180 mg, 93%): [α]_{D} +19.05 (c 0.3, MeOH); ¹H NMR (CD₃OD): δ 4.59 (br s, 1H, H-2), 4.31 (d, 1H, J_{3,2}=1.5 Hz, H-3), 4.03-3.97 (m, 1H, H-5a), 3.86-3.80 (m, 2H, H-4 and H-5b), 3.82 (d, 1H, J_{1a,1b}=12.0 Hz, H-1a), 3.68 (dd, 1H, J_{1b,1a}=12.0 Hz, J_{1b,2}=3.5 Hz, H-1b), 3.48 (ddd, 1H, J_{1′a,1′b}=12.5 Hz, J_{1′a,2′a}=J_{1′a,2′b}=8.0 Hz, H-1′a), 3.40 (ddd, 1H, J_{1′b,1′a}=12.5 Hz, J_{1′b,2′a}=8.5 Hz, J_{1′b,2′b}=6.0 Hz, H-1′b), 1.91-1.78 (m, 2H, H-2′), 1.55-1.42 (m, 2H, H-3′), 1.39-1.32 (m, 4H, H-4′ and H-5′), 0.90 (dd, 3H, J_{6′,5′a}=J_{6′,5′b}=7.5 Hz, H-6′); ¹³C NMR (CD₃OD): c) δ 79.6 (C-3), 79.4 (C-2), 73.6 (C-4), 61.0 (C-5), 50.2 (C-1), 46.7 (C-1′), 32.3 (1C, C-4′ or C-5′), 28.9 (C-3′), 26.8 (C-2′), 23.5 (1C, C-4′ or C-5′), 14.3 (C-6′); MALDI-TOF MS: m/z 235.21 [M−Cl]⁺. Anal. Calcd for C₁₁H₂₃ClO₃S: C, 48.78; H, 8.56. Found: C, 48.50; H, 8.76.

### 1,4-Dideoxy-1,4-[(1-octyl)-(R)-episulfoniumylidene]-D-arabinitol chloride (47)

Treatment of the sulfonium-ion 67 (500 mg, 0.81 mmol) with BCl₃ in CH₂Cl₂ (10 mL), followed by treatment with Amberlyst A-26 resin (250 mg) gave the final product 47 as a colorless syrup (238 mg, 99%): [α]_{D} +22.22 (c 0.3, MeOH); ¹H NMR (CD₃OD): δ 4.62 (br s, 1H, H-2), 4.34 (br s, 1H, H-3), 4.06-4.01 (m, 1H, H-5a), 3.89-3.83 (m, 2H, H-4 and H-5b), 3.85 (d, 1H, J_{1a,1b}=12.5 Hz, H-1a), 3.71 (dd, 1H, J_{1b,1a}=12.5 Hz, J_{1b,2}=2.5 Hz, H-1b), 3.50 (ddd, 1H, J_{1′a,1′b}=12.5 Hz, J_{1′a,2′a}=J_{1′a,2′b}=8.0 Hz, H-1′a), 3.43 (ddd, 1H, J_{1′b,1′a}=12.5 Hz, J_{1′b,2′a}=8.0 Hz, J_{1′b,2′b}=6.0 Hz, H-1′b), 1.91-1.83 (m, 2H, H-2′), 1.56-1.49 (m, 2H, H-3′), 1.46-1.32 (m, 8H, H-4′-H-7′) 0.90 (dd, 3H, J_{8′,7′a}=J_{8′,7′b}=6.0 Hz, H-8′); ¹³C NMR (CD₃OD): δ 79.6 (C-3), 79.4 (C-2), 73.6 (C-4), 61.0 (C-5), 50.1 (C-1), 46.7 (C-1′), 32.9, 30.1, 30.0 and 23.7 (4C, C-4′-C-7′), 29.2 (C-3′), 26.8 (C-2′), 14.5 (C-8′); MALDI-TOF MS: m/z 263.33 [M−Cl]⁺. Anal. Calcd for C₁₃H₂₇ClO₃S: C, 52.24; H, 9.11. Found: C, 52.55; H, 8.89.

### 1,4-Dideoxy-1,4-[(1-tetradecyl)-(R)-episulfoniumylidene]-D-arabinitol chloride (48)

Treatment of the sulfonium-ion 66 (200 mg, 0.28 mmol) with BCl₃ in CH₂Cl₂ (5 mL), followed by treatment with Amberlyst A-26 resin (150 mg) gave the final product 48 as a colorless, amorphous solid (120 mg, 98%): mp 86-88° C.; [α]_{D} +10.87 (c 0.2, MeOH); ¹H NMR (CD₃OD): δ 4.62 (br s, 1H, H-2), 4.34 (br s, 1H, H-3), 4.10-4.0 (m, 1H, H-5a), 3.90-3.83 (m, 2H, H-4 and H-5b), 3.84 (d, 1H, J_{1a,1b}=12.5 Hz, H-1a), 3.71 (dd, 1H, J_{1b,1a}=12.5 Hz, J_{1b,2}=3 Hz, H-1b), 3.50 (ddd, 1H, J_{1′a,1′b}=12.5 Hz, J_{1′a,2′a}=J_{1′a,2′b}=8.0 Hz, H-1′a), 3.42 (ddd, 1H, J_{1′b,1′a}=12.5 Hz, J_{1′b,2′a}=8.5 Hz, J_{1′b,2′b}=5.5 Hz, H-1′b), 1.91-1.81 (m, 2H, H-2′), 1.58-1.45 (m, 2H, H-3′),1.43-1.28 (m, 20H, H-4′-H-13′), 0.89 (dd, 3H, J_{14′,13′a}=J_{14′,13′b}=7.0 Hz, H-14′); ¹³C NMR (CD₃OD): δ 79.6 (C-3), 79.3 (C-2), 73.6 (C-4), 61.0 (C-5), 50.1 (C-1), 46.7 (C-1′), 33.1-23.8 (10C, C-4′-C-13′), 29.2 (C-3′), 26.8 (C-2′), 14.5 (C-14′); MALDI-TOF MS: m/z 347.12 [M−Cl]⁺. Anal. Calcd for C₁₉H₃₉ClO₃S: C, 59.58; H, 10.26. Found: C, 59.26; H, 10.33.

### 1,4-Dideoxy-1,4-[(1-octadecyl)-(R)-episulfoniumylidene]-D-arabiiiitol chloride (49)

Treatment of the sulfonium-ion 68 (450 mg, 0.59 mmol) with BCl₃ in CH₂Cl₂ (10 mL), followed by treatment with Amberlyst A-26 resin (280 mg) gave the final product 49 as a colorless amorphous solid (256 mg, 98%): mp 95-97° C.; [α9 _{D} +12.92 (c 0.5, MeOH); ¹H NMR (CD₃OD): δ 4.61 (br s, 1H, H-2), 4.33 (br s, 1H, H-3), 4.05-3.99 (m, 1H, H-5a), 3.88-3.82 (m, 2H, H-4 and H-5b), 3.83 (d, 1H, J_{1a,1b}=12.5 Hz, H-1a), 3.70 (dd, 1H, J_{1b,1a}=12.5 Hz, J_{1b,2}=3.2 Hz, H-1b), 3.49 (ddd, 1H, J_{1′a,1′b}=12.5 Hz, J_{1′a,2a}=J_{1′a,2′b}=8.0 Hz, H-1′a), 3.41 (ddd, 1H, J_{1′b,1′a}=12.5 Hz, J_{1′b,2′a}=8.5 Hz, J_{1′b,2′b}=6.0 Hz, H-1′b), 1.92-1.81 (m, 2H, H-2′), 1.56-1.42 (m, 2H, H-3′), 1.41-1.27 (m, 28H, H-4′-H-17′), 0.88 (dd, 3H, J_{18′,17′a}=J₁₈′,17′b=7.0 Hz, H-18′); ¹³C NMR (CD₃OD): δ 79.6 (C-3), 79.4 (C-2), 73.6 (C-4), 61.0 (C-5), 50.2 (C-1), 46.7 (C-1′), 33.1-23.8 (14C, C-4′-C-17′), 29.3 (C-3′), 26.8 (C-2′), 14.5 (C-18′); MALDI-TOF MS: m/z 403.47 [M−Cl]⁺. Anal. Calcd for C₂₃H₄₇ClO₃S: C, 62.91; H, 10.79. Found: C, 62.57; H, 10.41.

### 1,4-Dideoxy-1,4-[[1-(3-methyl)-butyl]-(R)-episulfoniumylidene]-D-arabinitol chloride (50)

Treatment of the sulfonium-ion 69 (340 mg, 0.59 mmol) with BCl₃ in CH₂Cl₂ (8 mL), followed by treatment with Amberlyst A-26 resin (160 mg) gave the final product 50 as a colorless syrup (144 mg, 95%): [α]_{D} +21.15 (c 0.4, MeOH); ¹H NMR (CD₃OD): δ 4.59 (br s, 1H, H-2), 4.31 (d, 1H, J_{3,2}=2.0 Hz, H-3), 4.03-3.97 (m, 1H, H-5a), 3.85-3.80 (m, 2H, H-4 and H-5b), 3.81 (d, 1H, J_{1a,1b}=12.0 Hz, H-1a), 3.67 (dd, 1H, J_{1b,1a}=12.0 Hz, J_{1b,2}=3.0 Hz, H-1b), 3.49 (ddd, 1H, J_{1′a,1′b}=12.5 Hz, J_{1′a,2′a}=J_{1′a,2′b}=8.0 Hz, H-1′a), 3.38 (ddd, 1H, J_{1′b,1′a}=12.5 Hz, J_{1′b,2′a}=J_{1′b,2′b}=7.5 Hz, H-1′b), 1.79-1.70 (m, 3H, H-2′ and H-3′), 0.96 (2d, each 3H, J_{4′,3′}=J_{5′,3′}=6.5 Hz, H-4′ and H-5′); ¹³C NMR (CD₃OD): δ 79.6 (C-3), 79.4 (C-2), 73.6 (C-4), 61.0 (C-5), 50.2 (C-1), 44.8 (C-1′), 35.3 (C-2′), 28.6 (C-3′), 22.5, 22.2 (2C, C-4′ and C-5′); MALDI-TOF MS: m/z 221.14 [M−Cl]⁺. Anal. Calcd for C₁₀H₂₁ClO₃S: C, 46.77; H, 8.24. Found: C, 46.43; H, 8.09.

### 1,4-Dideoxy-1,4-[[1-(6-ethoxy)-hexyl]-(R)-episulfoniumylidene]-D-arabinitol chloride (51)

Treatment of the sulfonium-ion 72 (230 mg, 0.36 mmol) with BCl₃ in CH₂Cl₂ (7 mL), followed by treatment with Amberlyst A-26 resin (150 mg) gave the final product 51 as a colorless syrup (110 mg, 97%): [α]_{D} +18.56 (c 0.3, MeOH); ¹H NMR (CD₃OD): δ 4.59 (br s, 1H, H-2), 4.30 (d, 1H, J_{3,2}=2.0 Hz, H-3), 4.03-3.97 (m, 1H, H-5a), 3.85-3.80 (m, 2H, H-4 and H-5b), 3.81 (d, 1H, J_{1a,1b}=12.5 Hz, H-1a), 3.68 (dd, 1H, J_{1b,1a}=12.5 Hz, J_{1b,2}=3.2 Hz, H-1b), 3.47 (ddd, 1H, J_{1′a,1′b}=12.5 Hz, J_{1′a,2′a}=J_{1′a,2′b}=8.0 Hz, H-1′a), 3.44 (q, 2H, J=7.0 Hz, —OCH₂CH₃), 3.41 (dd, 2H, J_{6′,5′a}=J_{6′,5′b}=6.5 Hz, H-6′), 3.40 (ddd, 1H, J_{1′b,1′a}=12.5 Hz, J_{1′b,2′a}=8.5 Hz, J_{′b,2′b}=6.0 Hz, H-1′b), 1.91-1.79 (m, 2H, H-2′), 1.59-1.52 (m, 2H, H-5′), 1.52-1.47 (m, 2H, H-3′), 1.44-1.39 (m, 2H, H-4′), 1.14 (t, 3H, J=7.0 Hz, —OCH₂CH₃); ¹³C NMR (CD₃OD): δ 79.6 (C-3), 79.5 (C-2), 73.7 (C-4), 71.5 (C-6′), 67.3 (1C, —OCH₂CH₃), 61.0 (C-5), 50.1 (C-1), 46.6 (C-¹′), 30.4 (C-5′), 29.0 (C-3′), 26.8 (C-2′), 26.7 (C-4′), 15.5 (1C, —OCH₂Cl₃); MALDI-TOF MS: m/z 279.20 [M−Cl]⁺. HRMS Calcd for C₁₃H₂₇ClO₄S: 279.1630 [M−Cl]⁺. Found: 279.1629.

### 1,4-Dideoxy-1,4-[[1-(9-methoxy)-nonyl]-(R)-episulfoniumylidene]-D-arabinitol chloride (52)

Treatment of the sulfonium-ion 73 (248 mg, 0.37 mmol) with BCl₃ in CH₂Cl₂ (7 mL), followed by treatment with Amberlyst A-26 resin (150 mg) gave the final product 52 as a colorless syrup (120 mg, 94%): [α]_{D} +20.34 (c 0.5, MeOH); ¹H NMR (CD₃OD): δ 4.59 (br s, 1H, H-2), 4.31 (d, 1H, J_{3,2}=2.0 Hz, H-3), 4.03-3.97 (m, 1H, H-5a), 3.86-3.80 (m, 2H, H-4 and H-5b), 3.82 (d, 1H, J_{1a,1b}=12.5 Hz, H-1a), 3.68 (dd, 1H, J_{1b,1a}=12.0 Hz, J_{1b,2}=3.0 Hz, H-1b), 3.48 (ddd, 1H, J_{1′a,1′b}=12.5 Hz, J_{1′a,2′a}=J_{1′a,2′b}=8.0 Hz, H-1′a), 3.40 (ddd, 1H, J_{1′b,1′a}=12.5 Hz, J_{1′b,2′a}=8.5 Hz, J_{1′b,2′b}=5.5 Hz, H-1′b), 3.35 (dd, 2H, J_{9′,8′a}=J_{9′,8′b}=6.5 Hz, H-9′), 3.33 (s, 3H, —OCH₃), 1.91-1.78 (m, 2H, H-2′), 1.53-1.47 (m, 4H, H-3′ and H-8′), 1.37-1.31 (m, 8H, H-4′-H-7′); ¹³C NMR (CD₃OD): δ 79.6 (C-3), 79.4 (C-2), 73.9 (C-9′), 73.5 (C-4), 61.0 (C-5), 58.8 (1C, —OCH₃), 50.2 (C-1), 46.7 (C-¹′), 30.6, 30.4, 30.3, 30.0, 29.2, 27.1 (6C, C-3′-C-8′), 26.8 (C-2′); MALDI-TOF MS: m/z 307.30 [M−Cl]⁺. Anal. Calcd for C₁₅H₃₁ClO₄S: C, 52.54; H, 9.11. Found: C, 52.75; H, 9.07.

5.4 Chain-Modified Analogues with Frame Shift of Sulfate Moiety

Enzyme Activity Assay. Analysis of MGA inhibition was performed using maltose as the substrate, and measuring the release of glucose. Reactions were carried out in 100 mM MES buffer pH 6.5 at 37° C. for 15 min. The reaction was stopped by boiling for 3 min. 20 μL aliquots were taken and added to 100 μL of glucose oxidase assay reagent (Sigma) in a 96-well plate. Reactions were allowed to proceed for 1 h and absorbance was measured at 450 nm to determine the amount of glucose produced by MGA activity in the reaction. One unit of activity is defined as the hydrolysis of one mole of maltose per minute. All reactions were performed in triplicate and absorbance measurements were averaged to give a final result.

Enzyme Kinetics. Kinetic parameters of recombinant MGA were determined using the glucose oxidase assay to follow the production of glucose upon addition of enzyme (15 nM) at increasing maltose concentrations (from 1 mM-3.5 mM) with a reaction time of 15 min. The program GraFit 4.0.14 was used to fit the data to the Michaelis-Menten equation and estimate the kinetic parameters, Kₘ and Vₘₐₓ, of the enzyme. Kᵢ values for each inhibitor were determined by measuring the rate of maltose hydrolysis by MGA at varying inhibitor concentrations. Data were plotted in Lineweaver-Burk plots (1/rate vs. 1/[substrate]) and Kᵢ values were determined by the equation Kᵢ=Kₘ[I]/(Vₘₐₓ)m−Kₘ, where “m” is the the slope of the line. The Kᵢ reported for each inhibitor was estimated by averaging the Kᵢ values obtained from each of the different inhibitor concentrations.

General experimental: Optical rotations were measured at 23° C. ¹H and ¹³C-NMR spectra were recorded at 500 and 125 MHz, respectively. All assignments were confirmed with the aid of two dimensional experiments (¹H-¹H COSY, HMQC and HMBC). Column chromatography was performed with Merck silica gel 60 (230-400 mesh). MALDI-TOF mass spectra were recorded on a perSeptive Biosystems Voyager-DE spectrometer, using 2,5-dihydroxybenzoic acid as a matrix.

2,3,5-Tri-O-p-methoxybenzyl-L-xylitol-1,4-cyclic sulfate (86). To a solution of 2,3,5-tri p-methoxybenzyl-L-xylitol (82) (11.2 g, 0.021 mol) and triethylamine (11.7 mL, 0.084 mol) in CH₂Cl₂ (150 mL) at 0° C., was added thionyl chloride (2.4 mL, 0.031 mol) dropwise and the reaction mixture was stirred for 30 min. The mixture was poured into ice-cold water and extracted with additional CH₂Cl₂ (300 mL). The combined organic layers were washed with brine solution and dried over Na₂SO₄. The solvent was removed and the residue was purified by flash column chromatography to give an inseparable mixture of two diastereomeric cyclic sulfites as a pale brown oil (8.8 g, 72%). The mixture of the cyclic sulfites was redissolved in a mixture of CH₃CN: CCl₄ (1:1, 100 mL) Sodium periodate (5.0 g, 0.02 mol) and RuCl₃ (100 Mg) were added followed by the addition of 50 mL of water. The mixture was stirred for 2 h and then filtered through a bed of silica and washed with CH₂Cl₂. The volatiles were removed and the residue was partitioned between EtOAc (200 mL) and H₂O (100 mL). The organic layer was washed with brine solution and dried over Na₂SO₄. The solvent was evaporated under reduced pressure to give a pale yellow syrup that was purified by flash chromatography to give 86 as a colorless oil (7.8 g, 86%). [α]_{D} −10.0° (c 0.4, CH₂Cl₂). ¹H-NMR (CDCl₃): δ 4.99 (1H, dd, J_{4,5a}=7.1, J_{4,5b}=6.4 Hz, H-4), 4.53-4.32 (6H, 3×CH₂-Ph), 4.75 (1H, d, J_{1a,1b}=13.0 Hz, H-1a), 4.23 (1H, ddd, J_{1b,2}=3.2 Hz, H-1b), 3.81 (6H, s, 2×—OCH₃), 3.83 (3H, s, —OCH₃), 3.70 (1H, d, J_{3,2}=3.1 Hz, H-3), 3.63 (1H, dd, J_{5a,5b}=9.8, H-5a), 3.48 (1H, dd, H-5b), 3.46 (1H, dd, H-2). ¹³C-NMR (CDCl₃): δ 159.9-114.0 (18C, 3×PMB), 78.6 (C-4), 73.6(C-3), 73.1, 73.0, 71.0 (3×—CH₂-Ph), 72.6 (C-2), 67.8 (C-1), 66.6 (C-5), 55.5 (3×—OMe). MALDI-MS: m/e 597.0 (M⁺+Na). Anal. Calcd for C₂₉H₃₄O₁₀S: C, 60.61; H, 5.96. Found: C, 60.32; H, 5.76.

1,4-Dideoxy-1,4[[2S,3S,4S]-2,3,5-trihydroxy-4-(sulfooxy)pentyl]-epi-selenoniumylidene]-D-arabinitol Inner salt (78). The thioether 84 (257 mg, 0.50 mmol), the cyclic sulfate 86 (342 mg, 0.59 mmol) and K₂CO₃ (35 mg) were added to HFIP (3 mL) and the reaction mixture was stirred in a sealed tube for 72 h at 70° C. The solvent was removed and the residue was purified by flash column chromatography (EtOAc:MeOH, 15:1) to afford the coupled product as a colorless foam (312 mg, 57%). The coupled product was redissolved in CH₂Cl₂ (2 mL), TFA (10 mL) was then added and the reaction mixture was stirred for 1 h at room temperature. The volatiles were removed under high vacuum and the residue was purified by column chromatography (EtOAc:MeOH:H₂O 10:3:1) to give 78 as an amorphous solid (81 mg, 77%). [α]_{D} +40.0° (c 0.2, H₂O). ¹H-NMR (D₂O): δ 4.57 (1H, ddd, J_{2,3}=3.4 Hz, H-2), 4.29 (1H, ddd, J_{4′,5b′}=4.8 Hz, H-4′), 4.25 (1H, dd, J_{3,4}=3.2 Hz, H-3), 4.17 (1H, ddd, J_{2′,1b′}=9.1 Hz, H-2′), 3.98 (1H, dd, H-5a), 3.95 (1H, ddd, H-4), 3.81-3.72 (6H, m, H-5b, H-1a, H-1b, H-1a′, H-3′, H-5a′), 3.70 (1H, dd, J_{5b′,5a′}=12.3 Hz, H-5b′), 3.67 (1H, dd, J_{1b′,1a′}32 13.2 Hz, H-1b′). ¹³C-NMR (D₂O): 5 79.1 (C-4′), 77.4 (C-3), 76.7 (C-2), 71.2 (C-3′), 69.4 (C-4), 67.1 (C-2′), 59.8 (C-5′), 59.2 (C-5), 49.5 (C-1′), 47.7 (C-1). MALDI-MS: m/e 387.1 (M⁺+Na), 365.0 (M⁺+H), 285.2 (M⁺+H−SO₃). Anal. Calcd for C₁₀H₂₀O₁₀S₂: C, 32.96; H, 5.53. Found: C, 32.79; H, 5.56.

1,4-Dideoxy-1,4[[2S,3S,4S]-2,3,5-trihydroxy-4-(sulfooxy)-pentyl]-epi-selenoniumylidene]-D-arabinitol Inner salt (79). The selenoether 85 (246 mg, 0.44 mmol), the cyclic sulfate 86 (305 mg, 0.53 mmol) and K₂CO₃ (35 mg) were added to HFIP (4 mL) and the mixture was stirred in a sealed tube for 48 h at 70° C. The solvent was removed and the residue was purified by column chromatography. The coupled product was obtained as colorless foam (352 mg, 70%). The selenonium salts were deprotected using TFA following the same procedure that was used for compound 78. Purification by flash column chromatography (EtOAc:MeOH:H₂O, 10:3:1) gave an amorphous solid (79) (98 mg, 76%). [α]_{D} +27.0° (c 1.0, H₂O). ¹H-NMR (D₂O): δ 4.63 (1H, dd, J_{2,3}=4.0, J_{2,1}=4.0, H-2),4.32 (1H, dd, J_{3,4}=4.7 Hz, H-3), 4.29 (1H, ddd, J_{4′,5b′}=4.5, H-4′), 4.19 (1H, ddd, J_{2′,1b′}=8.3 Hz, H-2′), 4.02 (1H, ddd, H-4), 3.95 (1H, dd, J_{5a,4}=5.0, J_{5a,5b}=12.5 Hz, H-5a), 3.79 (1H, dd, J_{5b,4}=9.1 Hz, H-5b), 3.76 (3H, m, H-1a′, H-5a′, H-3′), 3.69 (1H, dd, J_{5b′,5a′}=12.3 Hz, H-5b′), 3.68 (1H, dd, J_{1b′,1a′}=12.1 Hz, H-1b′), 3.64 (2H, d, H₂-1). ¹³C-NMR (D₂O): δ 79.5 (C-4′), 78.1 (C-3), 77.4 (C-2), 71.7 (C-3′), 68.8 (C-4), 66.9 (C-2′), 59.9 (C-5′), 59.3 (C-5), 47.4 (C-1′), 45.0 (C-1). MALDI-MS: m/e 412.4 (M⁺+H), 332.2 (M⁺+H−SO₃). Anal. Calcd for C₁₀H₂₀O₁₀SSe: C, 29.20; H, 4.90. Found: C, 28.80; H, 4.56.

2,3,5-Tri-O-p-methoxybenzyl-D-xylitol-1,4-cyclic sulfate (88). To a solution of 2,3,5-tri-O-p-methoxybenzyl-D-xylitol (82) (9.8 g, 0.019 mol) and triethylamine (10.6 mL, 0.076 mol) in CH₂Cl₂ (150 mL) at 0° C., was added thionyl chloride (2.2 mL, 0.028 mol) in CH₂Cl₂ (10 mL) dropwise. After stirring for 30 min, the mixture was poured into ice-cold water and extracted with additional CH₂Cl₂ (200 mL). The combined organic layers were washed with brine solution and dried over Na₂SO₄. The solvent was removed and the residue was purified by flash column chromatography to give an inseparable mixture of two diastereomeric cyclic sulfites as a pale brown oil (8.1 g, 76%). The cyclic sulfites were oxidized following the same procedure that was used for compound 86. The residue was purified by flash column chromatography to give 88 as a colorless oil (7.2 g, 86%). [α]_{D} +11.4° (c 1.24, CH₂Cl₂). ¹H-NMR (CDCl₃): δ 7.21-6.82 (12H, 3×PMB), 4.99 (1H, dd, J_{4,5a}=7.1, J_{4,5b}=6.4 Hz, H-4), 4.53-4.32 (6H, 3×CH₂-Ph), 4.75 (1H, d, J_{1a,1b}=13.0 Hz, H-1a), 4.23 (1H, ddd, J_{1b,2}=3.2 Hz, H-1b), 3.81 (6H, s, 2×—OCH₃), 3.83 (3H, s, —OCH₃), 3.70 (1H, d, J_{3,2}=3.1 Hz, H-3), 3.63 (1H, dd, J_{5a,5b}=9.8, H-5a), 3.48 (1H, dd, H-5b), 3.46 (1H, dd, H-2). ¹³C-NMR (CDCl₃): δ 159.9-114.0 (18C, 3×PMB), 78.6 (C-4), 73.6 (C-3), 73.1, 73.0, 71.0 (3×—CH₂-Ph), 72.6 (C-2), 67.8 (C-1), 66.6 (C-5), 55.5 (3×—OMe). MALDI-MS: m/e 597.2 (M⁺+Na). Anal. Calcd for C₂₉H₃₄O₁₀S: C, 60.61; H, 5.96. Found: C, 60.42; H, 5.86.

1,4-Dideoxy-1,4[[2R,3R,4R]-2,3,5-trihydroxy-4-(sulfooxy)-pentyl]-epi-sulfoniumylidene]-D-arabinitol Inner salt (80). The thioether (84) (212 mg, 0.41 mmol) was coupled to the cyclic sulfate 88 (290 mg, 0.50 mmol) in HFIP (3 mL) following the same procedure that was used for the synthesis of 78. The residue was purified by flash column chromatography (EtOAc:MeOH, 15:1) to give the sulfonium salt as an amorphous solid (240 mg, 53%). Deprotection of the sulfonium salt using TFA and purification by column chromatography (EtOAc:MeOH:H₂O, 10:3:1) gave 80 as an amorphous solid (67 mg, 83%). [α]_{D} −21.2° (c 0.8, H₂O). 1H-NMR (D₂O): δ 4.59 (1H, ddd, J_{2,3}=3.8, J_{2,1a}=4.1, J_{2,1b}=4.0, H-2), 4.30 (1H, dd, J_{3,4}=3.2 Hz, H-3), 4.28 (1H, ddd, H-4′), 4.17 (1H, ddd, H-2′), 3.95 (1H, ddd, J_{4,5a}=5.0, J_{4,5b}=7.1, H-4), 3.92 (1H, dd, H-5a), 3.79 (1H, dd, H-5b), 3.78-3.70 (6H, m, H-1a, H-1a′, H-1b′, H-5a′, H-5b′, H-3′), 3.36 (1H, dd, H-1b). ¹³C-NMR (D₂O): δ 79.1 (C-4′), 77.7 (C-3), 76.8 (C-2), 71.2 (C-3′), 69.8 (C-4), 67.3 (C-2′), 59.9 (C-5′), 59.2 (C-5), 49.1 (C-1′), 49.3 (C-1). MALDI-MS: m/e 387.1 (M⁺+Na), 365.2 (M⁺+H), 285.4 (M⁺+H−SO₃). Anal. Calcd for C₁₀H₂₀O₁₀S₂: C, 32.96; H, 5.53. Found: C, 32.58; H, 5.82.

1,4-Dideoxy-1,4[[2R,3R,4R]-2,3,5-trihydroxy-4-(sulfooxy)-pentyl]-epi-selenoniumylidene]-D-arabinitol Inner salt (81). The selenoether (85) (253 mg, 0.45 mmol) was coupled to the cyclic sulfate (88) (314 mg, 0.54) in HFIP (3 mL) following the same procedure that was used for the synthesis of 79. Column chromatography (EtOAc:MeOH, 20:1) of the crude product gave the selenonium salt as an amorphous solid (341 mg, 66%). Removal of the protecting groups and purification by column chromatography (EtOAc:MeOH:H₂O, 10:3:1) gave 81 as an amorphous solid (97 mg, 78%). [α]_{D} −95.0° (c 0.2, H₂O). ¹H-NMR (D₂O): δ 4.59 (1H, ddd, J_{2,3}=3.8, J_{2,1b}=4.1 Hz, H-2), 4.29 (1H, dd, J_{3,4}=3.8, H-4), 4.24 (1H, ddd, J_{4′,5b′}=4.8, J_{4′,5a′}=3.4, J_{4′,3′}=9.6 Hz, H-4′), 4.15 (1H, ddd, H-2′), 4.07 (1H, ddd, J_{4,5a}=5.1, J_{4,5b}=7.7 Hz, H-4), 3.94 (1H, dd, H-5a), 3.84 (1H, dd, H-5b), 3.80-3.73 (4H, m, H-1a′, H-1b′, H-3′, H-5a′), 3.69 (1H, dd, J_{5b′,5a′}=12.3, H-5b′), 3.65 (1H, dd, J_{1a,1b}=12.3 Hz, H-1a), 3.61 (1H, dd, H-1b). ¹³C-NMR (d₄-MeOH): δ 79.2 (C-4′), 78.8 (C-3), 78.2 (C-2), 71.8 (C-3′), 70.8 (C-4), 67.5 (C-2′), 60.0 (C-5′), 59.6 (C-5), 47.3 (C-1′), 45.0 (C-1). MALDI-MS: m/e 412.8 (M⁺+H), 332.6 (M⁺+H−SO₃). Anal. Calcd for C₁₀H₂₀O₁₀SSe: C, 29.20; H, 4.90. Found: C, 28.89; H, 4.82.

As will be apparent to those skilled in the art in the light of the foregoing disclosure, many alterations and modifications are possible in the practice of this invention without departing from the spirit or scope thereof.

### REFERENCES

1. Yoshikawa, M. et al. Tetrahedron Lett. 1997, 38(48), 8367-8370.
2. Yoshikawa, M. et al. Chem. Pharm. Bull. 1998, 46(8), 1339-1340.
3. Shimoda, H. et al. Journal of the Food Hygienic Society of Japan. 1999, 40(3), 198-205.
4. Yuasa, H.; Takada, J.; Hashimoto, H. Tetrahedron Lett. 2000, 41, 6615-6618.
5. Ghavami, A.; Johnston, B. D.; Pinto, B. M. J. Org. Chem. 2001, 66, 2312-2317.
6. Ghavami, A.; Johnston, B. D.; Maddess, M. D.; Chinapoo, S. M.; Jensen, M. T.; Svensson, B.; Pinto, B. M. Can. J. Chem. 2002, 80, 937-942.
7. Johnston, B. D.; Ghavami, A.; Jensen, M. T.; Svensson, B.; Pinto, B. M. J. Am. Chem. Soc. 2002, 124, 8245-8250.
8. Ghavami, A.; Johnston, B. D. ; Jensen, M. T. ; Svensson, B. ; Pinto, B. M.; J Am. Chem. Soc. 2001, 123, 6268-6271.
9. Pinto, B. M.; Johnston, B. D.; Ghavami, A.; Szczepina, M. G.; Lui, H.; Sadalapure, K., U.S. patent application Ser. No. 10/877,490 filed Jun. 25, 2004.
10. Yuasa, H.; Takada, J.; Hashimoto, H. Bioorg. Med. Chem. Lett. 2001, 11, 1137-1139.
11. Yuasa, H.; Saotome, C.; Kanie, O. Trends in Glycoscience and Glycotechnology 2002, 14, 231-251.
12. Kuntz, D. A.; Ghavami, A.; Johnston, B. D.; Pinto, B. M.; Rose, D. R. Tetrahedron: Asymmetry 2005, 16, 25-32.
13. Nakano, Y.; Maki, T.; Matsunaga, S.; van Soest, R. W. M.; Fusetani, N. Tetrahedron 2000, 56, 8977-8987.
14. Kafarski, P.; Lejczak, B. Phosphorous, Sulfur, Silicon 1991, 63, 193-215.
15. Winum, J.-Y.; Innocenti, A.; Gagnard, V.; Montero, J. -L.; Scozzafava, A.; Vullo, D.; Supuran, C. T. Bioorg. Med. Chem. Lett. 2005, 15, 1683-1686.
16. Tadeusiak, E. J. Bioorg. Chem. 2004, 32, 473-482.
17. Cushman, M.; Sambaiah, T.; Jin, G.; Illarionov, B.; Fischer, M.; Bacher, A. J. Org. Chem. 2004, 69, 601-612.
18. Elbein, A. D. Annu. Rev. Biochem. 1987, 56, 497-534.
19. Fellows, L. E.; Kite, G. C.; Nash, R. J.; Simmonds, M. S. J.; Scofield, A. M. Castanospermine, Swainsonine, and Related Polyhydroxy Alkaloids: Structure, Distribution and Biological Activity. In Plant Nitrogen Metabolism; Poulton, J. E.; Romero, J. T.; Conn, E. E., Eds.; Plenum: New York, 1989; pp 395-427.
20. Legler, G. Adv. Carbohydr. Chem. Biochem. 1990, 48, 319-384.
21. Stutz, A. E., Ed. Iminosugars as Glycosidase Inhibitors: Nojirimycin and Beyond; Wiley-VCH: Weinheim, New York, 1999.
22. Fosgerau, K.; Westergaard, N.; Quistorff, B.; Grunner, N.; Kristiansen, M.; Lundgren, K. Arch. Biochem. Biophys. 2000, 380, 274-284.
23. Fleet, G. W. J.; Nicholas, S. J.; Smith, P. W. Tetrahedron Lett. 1985, 26, 3127-3130.
24. Scofield, A. M.; Fellows, L. E.; Nash, R. J.; Fleet, G. W. J. Life Sci. 1986, 39, 645-650.
25. Bock, K.; Sigurskjold, B. Stud. Nat. Prod. Chem. 1990, 7, 29-86.
26. Holman, R. R.; Cull, C. A.; Turner, R. C. Diabetes Care 1999, 22, 960-964.
27. Jacob, G. S. Curr. Opin. Struct. Biol. 1995, 5, 605-611.
28. (a) Fleet, G. W. J.; Karpus, A.; Dwek, R. A.; Fellows, L. E.; Tyms, A. S.; Petursson, S.; Namgoong, S. K.; Ramsden, N. G.; Smith, P. W.; Son, J. C.; Wilson, F.; Witty, D. R.; Jacob, G. S.; Rademacher, T. W. FEBS Lett. 1988, 237, 128-132. (b) Karpas, A.; Fleet, G. W. J.; Dwek, R. A.; Petursson, S.; Namgoong, S. K.; Ramsden, N. G.; Jacob, G. S.; Rademacher, T. W. Proc. Natl. Acad. Sci. USA 1988, 85, 9229.
29. Schweden, J.; Borgmann, C.; Legler, G.; Bause, E. Arch. Biochem. Biophys. 1986, 248, 335-340.
30. van den Broek, L. A. G. M.; Vermaas, D. J.; Heskamp, B. M.; van Boeckel, C. A. A.; Tan, M. C. A. A.; Bolscher, J. G. M.; Ploegh, H. L.; van Kemenade, F. J.; de Goede, R. E. Y.; Miedema, F. Rec. Trav. Chim. Pays-Bas 1993, 112, 82-94.
31. van den Broek, L. A. G. M.; Vermaas, D. J.; van Kemenade, F. J.; Tan, M. C. A. A.; Rotteveel, F. T. M.; Zandberg, P.; Butters, T. D.; Miedema, F.; Ploegh, H. L.; van Boeckel, C. A. A. Rec. Trav. Chim. Pays-Bas 1994, 113, 507-516.
32. Mellor, H. R.; Nolan, J.; Pickering, L.; Wormald, M. R.; Platt, F. M.; Dwek, R. A.; Fleet, G. W. J.; Butters, T. D. Biochem. J. 2002, 366, 225-233.
33. Butters, T. D.; Van den Broek, L. A. G. M.; Fleet, G. W. J.; Krulle, T. M.; Wormald, M. R.; Dwek, R. A.; Platt, F. M. Tetrahedron: Asymmetry 2000, 11, 113-124.
34. (a) Sinnott, M. L. Chem. Rev. 1990, 90, 1171-1202. (b) Davies, G.; Sinnott, M. L.; Withers, S. G. In Comprehensive Biological Catalysis; Sinnott, M. L., Ed.; Academic Press: New York, 1997; Chapter 3, pp. 119-208 (c) Heightman, T. D.; Vasella, A. T. Angew. Chem. Int. Ed. Engl. 1999, 38, 750-770. (d) Withers, S. G.; Namchuk, M.; Mosi, R. In Iminosugars as Glycosidase inhibitors: Nojirimycin and Beyond; Stutz, A. E., Ed.; Wiley-VCH: Weinheim; New York, 1999; Chapter 9, pp. 188-206.
35. Goss, P. E. et al. Clinical Cancer Res. 1997, 3, 1077-1086.
36. Mohla, S. et al. Anticancer Res. 1990, 10, 1515-1522.
37. Goss, P. E. et al. Cancer Res. 1994, 54, 1450-1457.
38. Cimetiere, B.; Jacob, L.; Julia, M. Bull. Soc. Chim. Fr. 1991, 128, 926-938.
39. Bennis, K.; Calinaud, P.; Gelas, J.; Ghobsi, M. Carbohydr. Res. 1994, 264, 33-44.
40. Rao, B. V.; Lahiri, S. J. Carbohydr. Chem. 1996, 15, 975-984.
41. Sim, L.; Rose, D. R. unpublished observations.
42. Efange, S. M. N.; Michelson, R. H.; Dutta, A. K.; Parsons, S. M. J. Med. Chem. 1991, 34, 2638-2643.
43. Raic-Malic, S.; Svedruzic, D.; Gazivoda, T.; Marunovic, A.; Hergold-Brundic, A.; Nagl, A.; Balzarini, J.; De Clercq, E.; Mintas, M. J. Med. Chem. 2000, 43, 4806-4811.
44. Veerapen, N.; Yuan, Y.; Sanders, D. R. A.; Pinto, B. M. Carbohydr. Res. 2004, 339, 2205-2217.
45. Overkleeft, H. S.; Wiltenburg, J.; Pandit, U. K. Tetrahedron 1994, 50, 4215-4224.
46. Czarnocki, Z.; Mieczkowski, J. B.; Ziolkowski, M. Tetrahedron: Asymmetry 1996, 7, 2711-2720.
47. Andrews, G. C.; Crawford, T. C.; Bacon, B. E. J. Org. Chem. 1981, 46, 2976-2977.
48. Joseph, C. C.; Regeling, H.; Zwanenburg, B.; Chittenden, G. J. F. Tetrahedron 2002, 58, 6907-6912.
49. Kawatkar, S. P.; Kuntz, D. A.; Woods, R. J.; Rose, D. R.; Boons, G.-J. J. Am. Chem. Soc. 2006, ASAP ARTICLE.
50. Li, B.; Kawatkar, S. P.; George S; Strachan, H.; Woods, R. J.; Siriwardena, A.; Moremen, K. W.; Boons, G.-J. Chem Biochem 2004, 5, 1220-1127.
51. Satoh, H.; Yoshimura, Y.; Miura, S.; Machida, H. Bioorg. Med. Chem. Lett. 1998, 8, 989-992.
52. Girotra, N. N.; Biftu, T.; Ponpipom, M. M.; Acton, J. J.; Alberts, A. W. J. Med. Chem. 1992, 35, 3474-3482.
53. Doi, J. T.; Luehr, G. W. Tetrahedron Lett. 1985, 50, 6143-6146.
54. (a) Kumar, N. S.; Pinto, B. M. J. Org. Chem. 2006, 71, 1262-1264. (b) Kumar, N. S.; Pinto, B. M. Carbohydr. Res. 2006, 341, 1685-1691.
55. Rossi, E. J; Sim, L.; Kuntz, D. A.; Hahn, D.; Johnston, B. D.; Ghavami, A.; Szczepina, M. D.; Kumar, N. S.; Strerchi, E. E.; Nichols, B. L.; Pinto, B. M.; Rose, D. R. FEBS J. 2006, 273, 2673-2683.
56. Lui, H.; Pinto, B. M. J. Org. Chem. 2005, 70, 753-755.
57. Ghavami, A.; Sadalapure, K. S.; Johnston, B. D.; Lobera, M.; Snider, B. B.; Pinto, B. M. Synlett 2003, 1259-1262.
58. Johnston, B. D.; Jensen, H. H.; Pinto, B. M. J. Org. Chem. 2006, 71, 1111-1118.
59. Tanabe, G.; Yoshikai, Y.; Hatanaka, T.; Yamamoto, M.; Shao, Y.; Minematsu, T.; Muraoka, O.; Wang, T.; Matsuda, H.; Yoshikawa, M. Bio Org. Med. Chem. 2006, in press.
60. Liu, H.; Sim, L.; Rose, D. R.; Pinto, B. M. J. Org. Chem. 2006, 71, 3007-3013.
61. Brunger, A. T.; Adams, P. D.; Clore, G. M.; DeLano, W. L.; Gros, P.; Grosse-Kunstleve, R. W.; Jiang, J. S.; Kuszewski, J.; Nilges, M.; Pannu, N. S.; Read, R. J.; Rice, L. M.; Simonson, T.; Warren, G. L. Acta Crystallogr. D Biol Crystallogr. 1998, 54, 905-921.
62. Jones, T. A.; Zou, J. Y.; Cowan S. W.; Kjeldgaard, M. Acta Crystallogr. A 1991, 47,110-119.
63. Delano, W. L. The Py MOL Molecular Graphics System DeLano Scientific, San Carlos, Calif., 2002.
64. Cox, M.; Prager, R. H.; Svensson, C. E. Aust. J. Chem. 2003, 56, 887-896.

The above-listed documents are incorporated herein by reference.

What is claimed is:

1. A non-naturally occurring compound selected from the group consisting of compounds represented by the general formula (I) and pharmaceutically acceptable salts thereof: 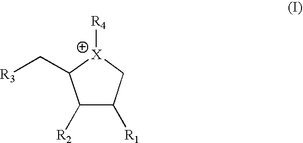
where X is selected from the group consisting of S, Se and NH; R₁, R₂, and R₃ are the same or different and are selected from the group consisting of H, OH, SH, NH₂ and halogens and R₄ is selected from the group consisting of:
(a) a polyhydroxylated acyclic alkyl chain comprising an anionic sulfate, carboxylate or phosphate moiety; and
(b) a lipophilic alkyl chain between 2 and 20 carbons in length with an external counterion.

2. The compound as defined in claim 1, wherein R₄ is an alditol side-chain.

3. The compound as defined in claim 1, wherein R₄ is a polyhydroxylated, acrylic chain comprising between 5 and 10 carbons.

4. The compound as defined in claim 3, wherein said chain comprises 5 or 6 carbons.

5. The compound as defined in claim 3, wherein X═S and wherein said compound is a chain-extended homologue of Salacinol.

6. The compound as defined in claim 1, wherein the heterocyclic ring is a D-arabinitol moiety.

7. The compound as defined in claim 6, wherein X═S or NH and R₄ is a polyhydroxylated five or six carbon chain having a terminal carboxylate residue.

8. The compound as defined in claim 6, wherein X═S or NH and R₄ is an alkyl chain having a terminal phosphate residue.

9. The compound as defined in claim 6, wherein X═S and R₄ comprises an alkoxy substitution at the end of said alkyl chain.

10. The compound as defined in claim 6, wherein X═S or Se and R₄ comprises a sulfate moiety located at carbon C-4′ of said acyclic chain.

11. A method of synthesizing a compound as defined in claims 7 or 8, comprising reacting a thioarabinitol or an imminoarabinitol with an epoxide to form a protected intermediate and deprotecting said intermediate.

12. A method of synthesizing a compound as defined in claim 10, comprising reacting a 7-membered cyclic sulfate with a thioarabinitol or selenoether to form a protected intermediate and deprotecting said intermediate.

13. The compound as defined in claim 1, wherein R₁, R₂ and R₃ are OH.

14. The use of the compound (I) of claim 1 for inhibiting the activity of a glucosidase enzyme.

15. The use as defined in claim 14, wherein said glycosidase enzyme is selected from the group consisting of intestinal maltase-glucoamylase, pancreatic alpha amylase and Golgi α-mannosidase II.

16. A pharmaceutical composition comprising an effective amount of a compound according to claim 1 together with a pharmaceutically acceptable carrier.

17. A method of treating a carbohydrate metabolic disorder in an affected patient comprising the step of administering to said patient a therapeutically effective amount of a compound according to claim 1.

18. The method of claim 17, wherein said carbohydrate metabolic disorder is non-insulin dependent diabetes.
